SEQUENCE LISTING (76 sequences)

SEQ ID NO: 1 - DNA, 24 bp, Artificial Sequence
  IR-A62
  misc_feature (1)..(2): Modifications in which methoxy or fluorine is introduced at the second carbon position or non-modification
  misc_feature (3)..(4): Modified Deoxyribose uracil in which the 5th carbon position is substituted with a hydrophobic group of a naphthyl group or a benzyl group
  misc_feature (5)..(9): Modifications in which methoxy or fluorine is introduced at the second carbon position or non-modification
  misc_feature (10)..(10): Modified Deoxyribose uracil in which the 5th carbon position is substituted with a hydrophobic group of a naphthyl group or a benzyl group
  misc_feature (11)..(13): Modifications in which methoxy or fluorine is introduced at the second carbon position or non-modification
  misc_feature (14)..(14): Modified Deoxyribose uracil in which the 5th carbon position is substituted with a hydrophobic group of a naphthyl group or a benzyl group
  misc_feature (15)..(15): Modifications in which methoxy or fluorine is introduced at the second carbon position or non-modification
  misc_feature (16)..(16): Modified Deoxyribose uracil in which the 5th carbon position is substituted with a hydrophobic group of a naphthyl group or a benzyl group
  misc_feature (17)..(19): Modifications in which methoxy or fluorine is introduced at the second carbon position or non-modification
  misc_feature (20)..(20): Modified Deoxyribose uracil in which the 5th carbon position is substituted with a hydrophobic group of a naphthyl group or a benzyl group
  misc_feature (21)..(23): Modifications in which methoxy or fluorine is introduced at the second carbon position or non-modification
  misc_feature (24)..(24): Modified Deoxyribose uracil in which the 5th carbon position is substituted with a hydrophobic group of a naphthyl group or a benzyl group
cannacgcangagncnaganccgn

SEQ ID NO: 2 - DNA, 79 bp, Artificial Sequence
  IR-A62
  misc_feature (22)..(23): Modifications in which methoxy or fluorine is introduced at the second carbon position or non-modification
  misc_feature (24)..(25): Modified Deoxyribose uracil in which the 5th carbon position is substituted with a hydrophobic group of a naphthyl group or a benzyl group
  misc_feature (26)..(30): Modifications in which methoxy or fluorine is introduced at the second carbon position or non-modification
  misc_feature (31)..(31): Modified Deoxyribose uracil in which the 5th carbon position is substituted with a hydrophobic group of a naphthyl group or a benzyl group
  misc_feature (32)..(34): Modifications in which methoxy or fluorine is introduced at the second carbon position or non-modification
  misc_feature (35)..(35): Modified Deoxyribose uracil in which the 5th carbon position is substituted with a hydrophobic group of a naphthyl group or a benzyl group
  misc_feature (36)..(36): Modifications in which methoxy or fluorine is introduced at the second carbon position or non-modification
  misc_feature (37)..(37): Modified Deoxyribose uracil in which the 5th carbon position is substituted with a hydrophobic group of a naphthyl group or a benzyl group
  misc_feature (38)..(40): Modifications in which methoxy or fluorine is introduced at the second carbon position or non-modification
  misc_feature (41)..(41): Modified Deoxyribose uracil in which the 5th carbon position is substituted with a hydrophobic group of a naphthyl group or a benzyl group
  misc_feature (42)..(44): Modifications in which methoxy or fluorine is introduced at the second carbon position or non-modification
  misc_feature (45)..(45): Modified Deoxyribose uracil in which the 5th carbon position is substituted with a hydrophobic group of a naphthyl group or a benzyl group
  misc_feature (52)..(52): n is a, c, g, or t
  misc_feature (58)..(59): n is a, c, g, or t
tatgagtgaccgtccgcctggcannacgcangagncnaganccgncagaccnaaggcnncagccacaccaccagccaaa

SEQ ID NO: 3 - DNA, 39 bp, Artificial Sequence
  IR-A62
  misc_feature (9)..(10): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (20)..(20): Modified Deoxyribose uracil in which the 5th carbon position is substituted with a hydrophobic group of a naphthylmethylcarboxamide
  misc_feature (22)..(22): Modified Deoxyribose uracil in which the 5th carbon position is substituted with a hydrophobic group of a naphthylmethylcarboxamide
  misc_feature (26)..(26): Modified Deoxyribose uracil in which the 5th carbon position is substituted with a hydrophobic group of a naphthylmethylcarboxamide
  misc_feature (30)..(30): Modified Deoxyribose uracil in which the 5th carbon position is substituted with a hydrophobic group of a naphthylmethylcarboxamide
  misc_feature (37)..(37): Modified Deoxyribose uracil in which the 5th carbon position is substituted with a hydrophobic group of a naphthylmethylcarboxamide
gcctggcannacgcangagncnaganccgncagaccnaa

SEQ ID NO: 4 - DNA, 27 bp, Artificial Sequence
  IR-A62
  misc_feature (2)..(2): Modifications in which fluorine is introduced at the second carbon position
  misc_feature (3)..(4): Modified Deoxyribose uracil in which the 5th carbon position is substituted with a hydrophobic group of a benzylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(13): Modifications in which methoxy is introduced at the second carbon position
  misc_feature (14)..(14): Modified Deoxyribose uracil in which the 5th carbon position is substituted with a hydrophobic group of a benzylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a benzylcarboxamide
  misc_feature (25)..(27): Modifications in which methoxy is introduced at the second carbon position
cannacgcangagncnaganccgncag

SEQ ID NO: 5 - DNA, 26 bp, Artificial Sequence
  IR-A62
  misc_feature (2)..(2): Modifications in which fluorine is introduced at the second carbon position
  misc_feature (3)..(4): Modified Deoxyribose uracil in which the 5th carbon position is substituted with a hydrophobic group of a benzylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(13): Modifications in which methoxy is introduced at the second carbon position
  misc_feature (14)..(14): Modified Deoxyribose uracil in which the 5th carbon position is substituted with a hydrophobic group of a benzylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a benzylcarboxamide
  misc_feature (25)..(26): Modifications in which methoxy is introduced at the second carbon position
cannacgcangagncnaganccgnca

SEQ ID NO: 6 - DNA, 25 bp, Artificial Sequence
  IR-A62
  misc_feature (2)..(2): Modifications in which fluorine is introduced at the second carbon position
  misc_feature (3)..(4): Modified Deoxyribose uracil in which the 5th carbon position is substituted with a hydrophobic group of a benzylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(13): Modifications in which methoxy is introduced at the second carbon position
  misc_feature (14)..(14): Modified Deoxyribose uracil in which the 5th carbon position is substituted with a hydrophobic group of a benzylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a benzylcarboxamide
  misc_feature (25)..(25): Modifications in which methoxy is introduced at the second carbon position
cannacgcangagncnaganccgnc

SEQ ID NO: 7 - DNA, 24 bp, Artificial Sequence
  IR-A62
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
cannacgcangagncnaganccgn

SEQ ID NO: 8 - DNA, 27 bp, Artificial Sequence
  IR-A62
  misc_feature (2)..(2): Modifications in which fluorine is introduced at the second carbon position
  misc_feature (3)..(4): Modified Deoxyribose uracil in which the 5th carbon position is substituted with a hydrophobic group of a benzylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a benzylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a benzylcarboxamide
  misc_feature (25)..(27): Modifications in which methoxy is introduced at the second carbon position
cannacgcangagncnaganccgncag

SEQ ID NO: 9 - DNA, 26 bp, Artificial Sequence
  IR-A62
  misc_feature (2)..(2): Modifications in which fluorine is introduced at the second carbon position
  misc_feature (3)..(4): Modified Deoxyribose uracil in which the 5th carbon position is substituted with a hydrophobic group of a benzylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a benzylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a benzylcarboxamide
  misc_feature (25)..(26): Modifications in which methoxy is introduced at the second carbon position
cannacgcangagncnaganccgnca

SEQ ID NO: 10 - DNA, 27 bp, Artificial Sequence
  IR-A62
  misc_feature (2)..(2): Modifications in which fluorine is introduced at the second carbon position
  misc_feature (3)..(4): Modified Deoxyribose uracil in which the 5th carbon position is substituted with a hydrophobic group of a benzylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a benzylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a benzylcarboxamide
  misc_feature (25)..(25): Modifications in which methoxy is introduced at the second carbon position
  misc_feature (26)..(26): Modifications in which fluorine is introduced at the second carbon position
  misc_feature (27)..(27): Modifications in which methoxy is introduced at the second carbon position
cannacgcangagncnaganccgncag

SEQ ID NO: 11 - DNA, 27 bp, Artificial Sequence
  IR-A62
  misc_feature (2)..(2): Modifications in which fluorine is introduced at the second carbon position
  misc_feature (3)..(4): Modified Deoxyribose uracil in which the 5th carbon position is substituted with a hydrophobic group of a benzylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which fluorine is introduced at the second carbon position
  misc_feature (12)..(12): Modifications in which methoxy is introduced at the second carbon position
  misc_feature (13)..(13): Modifications in which fluorine is introduced at the second carbon position
  misc_feature (14)..(14): Modified Deoxyribose uracil in which the 5th carbon position is substituted with a hydrophobic group of a benzylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a benzylcarboxamide
  misc_feature (25)..(25): Modifications in which methoxy is introduced at the second carbon position
  misc_feature (26)..(26): Modifications in which fluorine is introduced at the second carbon position
  misc_feature (27)..(27): Modifications in which methoxy is introduced at the second carbon position
cannacgcangagncnaganccgncag

SEQ ID NO: 12 - DNA, 8 bp, Artificial Sequence
  Linker; (6bp)-1
  misc_feature (1)..(2): Spacer
ttgtggcc

SEQ ID NO: 13 - DNA, 8 bp, Artificial Sequence
  Linker; (6bp)-2
  misc_feature (1)..(2): Spacer
ttggccac

SEQ ID NO: 14 - DNA, 10 bp, Artificial Sequence
  Linker; (8bp)-1
  misc_feature (1)..(2): Spacer
ttattacagc

SEQ ID NO: 15 - DNA, 10 bp, Artificial Sequence
  Linker; (8bp)-2
  misc_feature (1)..(2): Spacer
ttgctgtaat

SEQ ID NO: 16 - DNA, 12 bp, Artificial Sequence
  Linker; (10bp)-1
  misc_feature (1)..(2): Spacer
ttattacagctt

SEQ ID NO: 17 - DNA, 12 bp, Artificial Sequence
  Linker; (10bp)-2
  misc_feature (1)..(2): Spacer
ttaagctgtaat

SEQ ID NO: 18 - DNA, 14 bp, Artificial Sequence
  Linker; (12bp)-1
  misc_feature (1)..(2): Spacer
ttattacagcttgc

SEQ ID NO: 19 - DNA, 14 bp, Artificial Sequence
  Linker; (12bp)-2
  misc_feature (1)..(2): Spacer
ttgcaagctgtaat

SEQ ID NO: 20 - DNA, 16 bp, Artificial Sequence
  Linker; (14bp)-1
  misc_feature (1)..(2): Spacer
ttattacagcttgcta

SEQ ID NO: 21 - DNA, 16 bp, Artificial Sequence
  Linker; (14bp)-2
  misc_feature (1)..(2): Spacer
tttagcaagctgtaat

SEQ ID NO: 22 - DNA, 18 bp, Artificial Sequence
  Linker; (16bp)-1
  misc_feature (1)..(2): Spacer
ttattacagcttgctaca

SEQ ID NO: 23 - DNA, 18 bp, Artificial Sequence
  Linker; (16bp)-2
  misc_feature (1)..(2): Spacer
tttgtagcaagctgtaat

SEQ ID NO: 24 - DNA, 20 bp, Artificial Sequence
  Linker; (18bp)-1
  misc_feature (1)..(2): Spacer
ttattacagcttgctacacg

SEQ ID NO: 25 - DNA, 20 bp, Artificial Sequence
  Linker; (18bp)-2
  misc_feature (1)..(2): Spacer
ttcgtgtagcaagctgtaat

SEQ ID NO: 26 - DNA, 22 bp, Artificial Sequence
  Linker; (20bp)-1
  misc_feature (1)..(2): Spacer
ttattacagcttgctacacgaa

SEQ ID NO: 27 - DNA, 22 bp, Artificial Sequence
  Linker; (20bp)-2
  misc_feature (1)..(2): Spacer
ttttcgtgtagcaagctgtaat

SEQ ID NO: 28 - DNA, 32 bp, Artificial Sequence
  A62 D-1 (6bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(32): Linker; (6bp)-1
cannacgcangagncnaganccgnttgtggcc

SEQ ID NO: 29 - DNA, 32 bp, Artificial Sequence
  A62 D-2 (6bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(32): Linker; (6bp)-2
cannacgcangagncnaganccgnttggccac

SEQ ID NO: 30 - DNA, 34 bp, Artificial Sequence
  A62 D-1 (8bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(34): Linker; (8bp)-1
cannacgcangagncnaganccgnttattacagc

SEQ ID NO: 31 - DNA, 34 bp, Artificial Sequence
  A62 D-2 (8bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(34): Linker; (8bp)-2
cannacgcangagncnaganccgnttgctgtaat

SEQ ID NO: 32 - DNA, 36 bp, Artificial Sequence
  A62 D-1 (10bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(36): Linker; (10bp)-1
cannacgcangagncnaganccgnttattacagctt

SEQ ID NO: 33 - DNA, 36 bp, Artificial Sequence
  A62 D-2 (10bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(36): Linker; (10bp)-2
cannacgcangagncnaganccgnttaagctgtaat

SEQ ID NO: 34 - DNA, 38 bp, Artificial Sequence
  A62 D-1 (12bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(38): Linker; (12bp)-1
cannacgcangagncnaganccgnttattacagcttgc

SEQ ID NO: 35 - DNA, 38 bp, Artificial Sequence
  A62 D-2 (12bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(38): Linker; (12bp)-2
cannacgcangagncnaganccgnttgcaagctgtaat

SEQ ID NO: 36 - DNA, 40 bp, Artificial Sequence
  A62 D-1 (14bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(40): Linker; (14bp)-1
cannacgcangagncnaganccgnttattacagcttgcta

SEQ ID NO: 37 - DNA, 40 bp, Artificial Sequence
  A62 D-2 (14bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(40): Linker; (14bp)-2
cannacgcangagncnaganccgntttagcaagctgtaat

SEQ ID NO: 38 - DNA, 42 bp, Artificial Sequence
  A62 D-1 (16bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(42): Linker; (16bp)-1
cannacgcangagncnaganccgnttattacagcttgctaca

SEQ ID NO: 39 - DNA, 42 bp, Artificial Sequence
  A62 D-2 (16bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(42): Linker; (16bp)-2
cannacgcangagncnaganccgntttgtagcaagctgtaat

SEQ ID NO: 40 - DNA, 44 bp, Artificial Sequence
  A62 D-1 (18bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(44): Linker; (18bp)-1
cannacgcangagncnaganccgnttattacagcttgctacacg

SEQ ID NO: 41 - DNA, 44 bp, Artificial Sequence
  A62 D-2 (18bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(44): Linker; (18bp)-2
cannacgcangagncnaganccgnttcgtgtagcaagctgtaat

SEQ ID NO: 42 - DNA, 46 bp, Artificial Sequence
  A62 D-1 (20bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(46): Linker; (20bp)-1
cannacgcangagncnaganccgnttattacagcttgctacacgaa

SEQ ID NO: 43 - DNA, 46 bp, Artificial Sequence
  A62 D-2 (20bp)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(46): Linker; (20bp)-2
cannacgcangagncnaganccgnttttcgtgtagcaagctgtaat

SEQ ID NO: 44 - DNA, 46 bp, Artificial Sequence; identical to SEQ ID NO: 42
  LD1
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(46): Linker; (20bp)-1
  misc_feature (25)..(46): it is substituted with methoxy at the second carbon position
  misc_binding (46)..(46): It is attached with an idT(inverted deoxythymidylate)

SEQ ID NO: 45 - DNA, 46 bp, Artificial Sequence; identical to SEQ ID NO: 43
  LD2
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(26): Spacer
  misc_feature (25)..(46): Linker; (20bp)-2
  misc_feature (25)..(46): it is substituted with methoxy at the second carbon position
  misc_binding (46)..(46): It is attached with an idT(inverted deoxythymidylate)

SEQ ID NO: 46 - DNA, 2 bp, Artificial Sequence
  Spacer; SSD (2T)
tt

SEQ ID NO: 47 - DNA, 4 bp, Artificial Sequence
  Spacer; SSD (4T)
tttt

SEQ ID NO: 48 - DNA, 6 bp, Artificial Sequence
  Spacer; SSD (6T)
tttttt

SEQ ID NO: 49 - DNA, 8 bp, Artificial Sequence
  Spacer; SSD (8T)
tttttttt

SEQ ID NO: 50 - DNA, 10 bp, Artificial Sequence
  Spacer; SSD (10T)
tttttttttt

SEQ ID NO: 51 - DNA, 15 bp, Artificial Sequence
  Spacer; SSD (15T)
ttttttttttttttt

SEQ ID NO: 52 - DNA, 20 bp, Artificial Sequence
  Spacer; SSD (20T)
tttttttttttttttttttt

SEQ ID NO: 53 - DNA, 1 bp, Artificial Sequence
  Spacer; SSD (C3 phosphoramidite)
  misc_feature (1)..(1): It is (Spacer Phosphoramidite C3)1
n

SEQ ID NO: 54 - DNA, 1 bp, Artificial Sequence
  Spacer; SSD (TEG phosphoramidite)
  misc_feature (1)..(1): It is (Spacer Phosphoramidite C 9)1
n

SEQ ID NO: 55 - DNA, 1 bp, Artificial Sequence
  Spacer; SSD (HEG1 phosphoramidite)
  misc_feature (1)..(1): It is (Spacer Phosphoramidite C 18)1
n

SEQ ID NO: 56 - DNA, 1 bp, Artificial Sequence
  Spacer; SSD (HEG2 phosphoramidite)
  misc_feature (1)..(1): It is (Spacer Phosphoramidite C 18)2
n

SEQ ID NO: 57 - DNA, 1 bp, Artificial Sequence
  Spacer; SSD (HEG3 phosphoramidite)
  misc_feature (1)..(1): It is (Spacer Phosphoramidite C 18)3
n

SEQ ID NO: 58 - DNA, 1 bp, Artificial Sequence
  Spacer; SSD (HEG4 phosphoramidite)
  misc_feature (1)..(1): It is (Spacer Phosphoramidite C 18)4
n

SEQ ID NO: 59 - DNA, 1 bp, Artificial Sequence
  Spacer; SSD (HEG5 phosphoramidite)
  misc_feature (1)..(1): It is (Spacer Phosphoramidite C 18)5
n

SEQ ID NO: 60 - DNA, 1 bp, Artificial Sequence
  Spacer; SSD (HEG6 phosphoramidite)
  misc_feature (1)..(1): (Spacer Phosphoramidite C 18)6
n

SEQ ID NO: 61 - DNA, 52 bp, Artificial Sequence
  A62 SSD (0T)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): it is substituted with methoxy at the second carbon position
  misc_feature (30)..(30): Modifications in which fluorine is introduced at the second carbon position
  misc_feature (31)..(32): Modified Deoxyribose uracil in which the 5th carbon position is substituted with a hydrophobic group of a naphthylmethylcarboxamide
  misc_feature (34)..(34): Modifications in which fluorine is introduced at the second carbon position
  misc_feature (36)..(36): Modifications in which fluorine is introduced at the second carbon position
  misc_feature (38)..(38): Modified Deoxyribose uracil in which the 5th carbon position is substituted with a hydrophobic group of a naphthylmethylcarboxamide
  misc_feature (39)..(39): Modifications in which methoxy is introduced at the second carbon position
  misc_feature (40)..(40): Modifications in which fluorine is introduced at the second carbon position
  misc_feature (41)..(41): Modifications in which methoxy is introduced at the second carbon position
  misc_feature (42)..(42): Modified Deoxyribose uracil in which the 5th carbon position is substituted with a hydrophobic group of a naphthylmethylcarboxamide
  misc_feature (44)..(44): Modified Deoxyribose uracil in which the 5th carbon position is substituted with a hydrophobic group of a naphthylmethylcarboxamide
  misc_feature (47)..(47): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (52)..(52): Modified Deoxyribose uracil in which the 5th carbon position is substituted with a hydrophobic group of a naphthylmethylcarboxamide
cannacgcangagncnaganccgnttttcannacgcangagncnaganccgn

SEQ ID NO: 62 - DNA, 54 bp, Artificial Sequence
  A62 SSD (2T)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(30): It is substituted with methoxy at the second carbon position
  misc_feature (27)..(28): Spacer; SSD (2T)
  misc_feature (32)..(32): Modifications in which fluorine is introduced at the second carbon position
  misc_feature (33)..(34): Modified Deoxyribose uracil in which the 5th carbon position is substituted with a hydrophobic group of a naphthylmethylcarboxamide
  misc_feature (36)..(36): Modifications in which fluorine is introduced at the second carbon position
  misc_feature (38)..(38): Modifications in which fluorine is introduced at the second carbon position
  misc_feature (40)..(40): Modified Deoxyribose uracil in which the 5th carbon position is substituted with a hydrophobic group of a naphthylmethylcarboxamide
  misc_feature (41)..(41): Modifications in which methoxy is introduced at the second carbon position
  misc_feature (42)..(42): Modifications in which fluorine is introduced at the second carbon position
  misc_feature (43)..(43): Modifications in which methoxy is introduced at the second carbon position
  misc_feature (44)..(44): Modified Deoxyribose uracil in which the 5th carbon position is substituted with a hydrophobic group of a naphthylmethylcarboxamide
  misc_feature (46)..(46): Modified Deoxyribose uracil in which the 5th carbon position is substituted with a hydrophobic group of a naphthylmethylcarboxamide
  misc_feature (49)..(49): Modifications in which fluorine is introduced at the second carbon position
  misc_feature (50)..(50): Modified Deoxyribose uracil in which the 5th carbon position is substituted with a hydrophobic group of a naphthylmethylcarboxamide
  misc_feature (51)..(51): Modifications in which methoxy is introduced at the second carbon position
  misc_feature (52)..(52): Modifications in which fluorine is introduced at the second carbon position
  misc_feature (54)..(54): Modified Deoxyribose uracil in which the 5th carbon position is substituted with a hydrophobic group of a naphthylmethylcarboxamide
cannacgcangagncnaganccgnttttttcannacgcangagncnaganccgn

SEQ ID NO: 63 - DNA, 56 bp, Artificial Sequence
  A62 SSD (4T)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(32): It is substituted with methoxy at the second carbon position
  misc_feature (27)..(30): Spacer; (4T)
  misc_feature (34)..(34): Modifications in which fluorine is introduced at the second carbon position
  misc_feature (35)..(36): Modified Deoxyribose uracil in which the 5th carbon position is substituted with a hydrophobic group of a naphthylmethylcarboxamide
  misc_feature (38)..(38): Modifications in which fluorine is introduced at the second carbon position
  misc_feature (40)..(40): Modifications in which fluorine is introduced at the second carbon position
  misc_feature (42)..(42): Modified Deoxyribose uracil in which the 5th carbon position is substituted with a hydrophobic group of a naphthylmethylcarboxamide
  misc_feature (43)..(43): Modifications in which methoxy is introduced at the second carbon position
  misc_feature (44)..(44): Modifications in which fluorine is introduced at the second carbon position
  misc_feature (45)..(45): Modifications in which methoxy is introduced at the second carbon position
  misc_feature (46)..(46): Modified Deoxyribose uracil in which the 5th carbon position is substituted with a hydrophobic group of a naphthylmethylcarboxamide
  misc_feature (48)..(48): Modified Deoxyribose uracil in which the 5th carbon position is substituted with a hydrophobic group of a naphthylmethylcarboxamide
  misc_feature (51)..(51): Modifications in which fluorine is introduced at the second carbon position
  misc_feature (52)..(52): Modified Deoxyribose uracil in which the 5th carbon position is substituted with a hydrophobic group of a naphthylmethylcarboxamide
  misc_feature (53)..(53): Modifications in which methoxy is introduced at the second carbon position
  misc_feature (54)..(54): Modifications in which fluorine is introduced at the second carbon position
  misc_feature (56)..(56): Modified Deoxyribose uracil in which the 5th carbon position is substituted with a hydrophobic group of a naphthylmethylcarboxamide
cannacgcangagncnaganccgnttttttttcannacgcangagncnaganccgn

SEQ ID NO: 64 - DNA, 58 bp, Artificial Sequence
  A62 SSD (6T)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(34): It is substituted with methoxy at the second carbon position
  misc_feature (27)..(32): Spacer; SSD (6T)
  misc_feature (36)..(36): Modifications in which fluorine is introduced at the second carbon position
  misc_feature (37)..(38): Modified Deoxyribose uracil in which the 5th carbon position is substituted with a hydrophobic group of a naphthylmethylcarboxamide
  misc_feature (40)..(40): Modifications in which fluorine is introduced at the second carbon position
  misc_feature (42)..(42): Modifications in which fluorine is introduced at the second carbon position
  misc_feature (44)..(44): Modified Deoxyribose uracil in which the 5th carbon position is substituted with a hydrophobic group of a naphthylmethylcarboxamide
  misc_feature (45)..(45): Modifications in which methoxy is introduced at the second carbon position
  misc_feature (46)..(46): Modifications in which fluorine is introduced at the second carbon position
  misc_feature (47)..(47): Modifications in which methoxy is introduced at the second carbon position
  misc_feature (48)..(48): Modified Deoxyribose uracil in which the 5th carbon position is substituted with a hydrophobic group of a naphthylmethylcarboxamide
  misc_feature (50)..(50): Modified Deoxyribose uracil in which the 5th carbon position is substituted with a hydrophobic group of a naphthylmethylcarboxamide
  misc_feature (53)..(53): Modifications in which fluorine is introduced at the second carbon position
  misc_feature (54)..(54): Modified Deoxyribose uracil in which the 5th carbon position is substituted with a hydrophobic group of a naphthylmethylcarboxamide
  misc_feature (55)..(55): Modifications in which methoxy is introduced at the second carbon position
  misc_feature (56)..(56): Modifications in which fluorine is introduced at the second carbon position
  misc_feature (58)..(58): Modified Deoxyribose uracil in which the 5th carbon position is substituted with a hydrophobic group of a naphthylmethylcarboxamide
cannacgcangagncnaganccgnttttttttttcannacgcangagncnaganccgn

SEQ ID NO: 65 - DNA, 60 bp, Artificial Sequence
  A62 SSD (8T)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(36): It is substituted with methoxy at the second carbon position
  misc_feature (27)..(34): Spacer; SSD (8T)
  misc_feature (38)..(38): Modifications in which fluorine is introduced at the second carbon position
  misc_feature (39)..(40): Modified Deoxyribose uracil in which the 5th carbon position is substituted with a hydrophobic group of a naphthylmethylcarboxamide
  misc_feature (42)..(42): Modifications in which fluorine is introduced at the second carbon position
  misc_feature (44)..(44): Modifications in which fluorine is introduced at the second carbon position
  misc_feature (46)..(46): Modified Deoxyribose uracil in which the 5th carbon position is substituted with a hydrophobic group of a naphthylmethylcarboxamide
  misc_feature (47)..(47): Modifications in which methoxy is introduced at the second carbon position
  misc_feature (48)..(48): Modifications in which fluorine is introduced at the second carbon position
  misc_feature (49)..(49): Modifications in which methoxy is introduced at the second carbon position
  misc_feature (50)..(50): Modified Deoxyribose uracil in which the 5th carbon position is substituted with a hydrophobic group of a naphthylmethylcarboxamide
  misc_feature (52)..(52): Modified Deoxyribose uracil in which the 5th carbon position is substituted with a hydrophobic group of a naphthylmethylcarboxamide
  misc_feature (55)..(55): Modifications in which fluorine is introduced at the second carbon position
  misc_feature (56)..(56): Modified Deoxyribose uracil in which the 5th carbon position is substituted with a hydrophobic group of a naphthylmethylcarboxamide
  misc_feature (57)..(57): Modifications in which methoxy is introduced at the second carbon position
  misc_feature (58)..(58): Modifications in which fluorine is introduced at the second carbon position
  misc_feature (60)..(60): Modified Deoxyribose uracil in which the 5th carbon position is substituted with a hydrophobic group of a naphthylmethylcarboxamide
cannacgcangagncnaganccgnttttttttttttcannacgcangagncnaganccgn

SEQ ID NO: 66 - DNA, 62 bp, Artificial Sequence
  A62 SSD (10T)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(38): It is substituted with methoxy at the second carbon position
  misc_feature (27)..(36): Spacer; SSD (10T)
  misc_feature (40)..(40): Modifications in which fluorine is introduced at the second carbon position
  misc_feature (41)..(42): Modified Deoxyribose uracil in which the 5th carbon position is substituted with a hydrophobic group of a naphthylmethylcarboxamide
  misc_feature (44)..(44): Modifications in which fluorine is introduced at the second carbon position
  misc_feature (46)..(46): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (51)..(51): Modifications in which methoxy is introduced at the second carbon position
  misc_feature (52)..(52): Modified Deoxyribose uracil in which the 5th carbon position is substituted with a hydrophobic group of a naphthylmethylcarboxamide
  misc_feature (54)..(54): Modified Deoxyribose uracil in which the 5th carbon position is substituted with a hydrophobic group of a naphthylmethylcarboxamide
  misc_feature (57)..(57): Modifications in which fluorine is introduced at the second carbon position
  misc_feature (58)..(58): Modified Deoxyribose uracil in which the 5th carbon position is substituted with a hydrophobic group of a naphthylmethylcarboxamide
  misc_feature (59)..(59): Modifications in which methoxy is introduced at the second carbon position
  misc_feature (60)..(60): Modifications in which fluorine is introduced at the second carbon position
  misc_feature (62)..(62): Modified Deoxyribose uracil in which the 5th carbon position is substituted with a hydrophobic group of a naphthylmethylcarboxamide
cannacgcangagncnaganccgnttttttttttttttcannacgcangagncnaganccgn

SEQ ID NO: 67 - DNA, 67 bp, Artificial Sequence
  A62 SSD (15T)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(43): It is substituted with methoxy at the second carbon position
  misc_feature (27)..(41): Spacer; SSD (15T)
  misc_feature (45)..(45): Modifications in which fluorine is introduced at the second carbon position
  misc_feature (46)..(47): Modified Deoxyribose uracil in which the 5th carbon position is substituted with a hydrophobic group of a naphthylmethylcarboxamide
  misc_feature (49)..(49): Modifications in which fluorine is introduced at the second carbon position
  misc_feature (51)..(51): Modifications in which fluorine is introduced at the second carbon position
  misc_feature (53)..(53): Modified Deoxyribose uracil in which the 5th carbon position is substituted with a hydrophobic group of a naphthylmethylcarboxamide
  misc_feature (54)..(54): Modifications in which methoxy is introduced at the second carbon position
  misc_feature (55)..(55): Modifications in which fluorine is introduced at the second carbon position
  misc_feature (56)..(56): Modifications in which methoxy is introduced at the second carbon position
  misc_feature (57)..(57): Modified Deoxyribose uracil in which the 5th carbon position is substituted with a hydrophobic group of a naphthylmethylcarboxamide
  misc_feature (59)..(59): Modified Deoxyribose uracil in which the 5th carbon position is substituted with a hydrophobic group of a naphthylmethylcarboxamide
  misc_feature (62)..(62): Modifications in which fluorine is introduced at the second carbon position
  misc_feature (63)..(63): Modified Deoxyribose uracil in which the 5th carbon position is substituted with a hydrophobic group of a naphthylmethylcarboxamide
  misc_feature (64)..(64): Modifications in which methoxy is introduced at the second carbon position
  misc_feature (65)..(65): Modifications in which fluorine is introduced at the second carbon position
  misc_feature (67)..(67): Modified Deoxyribose uracil in which the 5th carbon position is substituted with a hydrophobic group of a naphthylmethylcarboxamide
cannacgcangagncnaganccgntttttttttttttttttttcannacgcangagncnaganccgn

SEQ ID NO: 68 - DNA, 72 bp, Artificial Sequence
  A62 SSD (20T)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(48): It is substituted with methoxy at the second carbon position
  misc_feature (27)..(46): Spacer; SSD (20T)
  misc_feature (50)..(50): Modifications in which fluorine is introduced at the second carbon position
  misc_feature (51)..(52): Modified Deoxyribose uracil in which the 5th carbon position is substituted with a hydrophobic group of a naphthylmethylcarboxamide
  misc_feature (54)..(54): Modifications in which fluorine is introduced at the second carbon position
  misc_feature (56)..(56): Modifications in which fluorine is introduced at the second carbon position
  misc_feature (58)..(58): Modified Deoxyribose uracil in which the 5th carbon position is substituted with a hydrophobic group of a naphthylmethylcarboxamide
  misc_feature (59)..(59): Modifications in which methoxy is introduced at the second carbon position
  misc_feature (60)..(60): Modifications in which fluorine is introduced at the second carbon position
  misc_feature (61)..(61): Modifications in which methoxy is introduced at the second carbon position
  misc_feature (62)..(62): Modified Deoxyribose uracil in which the 5th carbon position is substituted with a hydrophobic group of a naphthylmethylcarboxamide
  misc_feature (64)..(64): Modified Deoxyribose uracil in which the 5th carbon position is substituted with a hydrophobic group of a naphthylmethylcarboxamide
  misc_feature (67)..(67): Modifications in which fluorine is introduced at the second carbon position
  misc_feature (68)..(68): Modified Deoxyribose uracil in which the 5th carbon position is substituted with a hydrophobic group of a naphthylmethylcarboxamide
  misc_feature (69)..(69): Modifications in which methoxy is introduced at the second carbon position
  misc_feature (70)..(70): Modifications in which fluorine is introduced at the second carbon position
  misc_feature (72)..(72): Modified Deoxyribose uracil in which the 5th carbon position is substituted with a hydrophobic group of a naphthylmethylcarboxamide
cannacgcangagncnaganccgnttttttttttttttttttttttttcannacgcangagncnaganccgn

SEQ ID NO: 69 - DNA, 52 bp, Artificial Sequence; identical to SEQ ID NO: 61
  A62 SSD (C3)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): It is substituted with methoxy at the second carbon position
  misc_binding (26)..(27): The two bases are linked each other with a (Spacer Phosphoramidite C3)1
  misc_feature (30)..(30): Modifications in which fluorine is introduced at the second carbon position
  misc_feature (31)..(32): Modified Deoxyribose uracil in which the 5th carbon position is substituted with a hydrophobic group of a naphthylmethylcarboxamide
  misc_feature (34)..(34): Modifications in which fluorine is introduced at the second carbon position
  misc_feature (36)..(36): Modifications in which fluorine is introduced at the second carbon position
  misc_feature (38)..(38): Modified Deoxyribose uracil in which the 5th carbon position is substituted with a hydrophobic group of a naphthylmethylcarboxamide
  misc_feature (39)..(39): Modifications in which methoxy is introduced at the second carbon position
  misc_feature (40)..(40): Modifications in which fluorine is introduced at the second carbon position
  misc_feature (41)..(41): Modifications in which methoxy is introduced at the second carbon position
  misc_feature (42)..(42): Modified Deoxyribose uracil in which the 5th carbon position is substituted with a hydrophobic group of a naphthylmethylcarboxamide
  misc_feature (44)..(44): Modified Deoxyribose uracil in which the 5th carbon position is substituted with a hydrophobic group of a naphthylmethylcarboxamide
  misc_feature (47)..(47): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (52)..(52): Modified Deoxyribose uracil in which the 5th carbon position is substituted with a hydrophobic group of a naphthylmethylcarboxamide

SEQ ID NO: 70 - DNA, 52 bp, Artificial Sequence; identical to SEQ ID NO: 61
  A62 SSD (TEG)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): It is substituted with methoxy at the second carbon position
  misc_binding (26)..(27): The two bases are linked each other with a (Spacer Phosphoramidite 9)1
  misc_feature (30)..(30): Modifications in which fluorine is introduced at the second carbon position
  misc_feature (31)..(32): Modified Deoxyribose uracil in which the 5th carbon position is substituted with a hydrophobic group of a naphthylmethylcarboxamide
  misc_feature (34)..(34): Modifications in which fluorine is introduced at the second carbon position
  misc_feature (36)..(36): Modifications in which fluorine is introduced at the second carbon position
  misc_feature (38)..(38): Modified Deoxyribose uracil in which the 5th carbon position is substituted with a hydrophobic group of a naphthylmethylcarboxamide
  misc_feature (39)..(39): Modifications in which methoxy is introduced at the second carbon position
  misc_feature (40)..(40): Modifications in which fluorine is introduced at the second carbon position
  misc_feature (41)..(41): Modifications in which methoxy is introduced at the second carbon position
  misc_feature (42)..(42): Modified Deoxyribose uracil in which the 5th carbon position is substituted with a hydrophobic group of a naphthylmethylcarboxamide
  misc_feature (44)..(44): Modified Deoxyribose uracil in which the 5th carbon position is substituted with a hydrophobic group of a naphthylmethylcarboxamide
  misc_feature (47)..(47): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (52)..(52): Modified Deoxyribose uracil in which the 5th carbon position is substituted with a hydrophobic group of a naphthylmethylcarboxamide

SEQ ID NO: 71 - DNA, 52 bp, Artificial Sequence; identical to SEQ ID NO: 61
  A62 SSD (HEG1)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): It is substituted with methoxy at the second carbon position
  misc_binding (26)..(27): The two bases are linked each other with a (Spacer Phosphoramidite 18)1
  misc_feature (30)..(30): Modifications in which fluorine is introduced at the second carbon position
  misc_feature (31)..(32): Modified Deoxyribose uracil in which the 5th carbon position is substituted with a hydrophobic group of a naphthylmethylcarboxamide
  misc_feature (34)..(34): Modifications in which fluorine is introduced at the second carbon position
  misc_feature (36)..(36): Modifications in which fluorine is introduced at the second carbon position
  misc_feature (38)..(38): Modified Deoxyribose uracil in which the 5th carbon position is substituted with a hydrophobic group of a naphthylmethylcarboxamide
  misc_feature (39)..(39): Modifications in which methoxy is introduced at the second carbon position
  misc_feature (40)..(40): Modifications in which fluorine is introduced at the second carbon position
  misc_feature (41)..(41): Modifications in which methoxy is introduced at the second carbon position
  misc_feature (42)..(42): Modified Deoxyribose uracil in which the 5th carbon position is substituted with a hydrophobic group of a naphthylmethylcarboxamide
  misc_feature (44)..(44): Modified Deoxyribose uracil in which the 5th carbon position is substituted with a hydrophobic group of a naphthylmethylcarboxamide
  misc_feature (47)..(47): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (52)..(52): Modified Deoxyribose uracil in which the 5th carbon position is substituted with a hydrophobic group of a naphthylmethylcarboxamide

SEQ ID NO: 72 - DNA, 52 bp, Artificial Sequence; identical to SEQ ID NO: 61
  A62 SSD (HEG2)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): It is substituted with methoxy at the second carbon position
  misc_binding (26)..(27): The two bases are linked each other with (Spacer Phosphoramidite 18)2
  misc_feature (30)..(30): Modifications in which fluorine is introduced at the second carbon position
  misc_feature (31)..(32): Modified Deoxyribose uracil in which the 5th carbon position is substituted with a hydrophobic group of a naphthylmethylcarboxamide
  misc_feature (34)..(34): Modifications in which fluorine is introduced at the second carbon position
  misc_feature (36)..(36): Modifications in which fluorine is introduced at the second carbon position
  misc_feature (38)..(38): Modified Deoxyribose uracil in which the 5th carbon position is substituted with a hydrophobic group of a naphthylmethylcarboxamide
  misc_feature (39)..(39): Modifications in which methoxy is introduced at the second carbon position
  misc_feature (40)..(40): Modifications in which fluorine is introduced at the second carbon position
  misc_feature (41)..(41): Modifications in which methoxy is introduced at the second carbon position
  misc_feature (42)..(42): Modified Deoxyribose uracil in which the 5th carbon position is substituted with a hydrophobic group of a naphthylmethylcarboxamide
  misc_feature (44)..(44): Modified Deoxyribose uracil in which the 5th carbon position is substituted with a hydrophobic group of a naphthylmethylcarboxamide
  misc_feature (47)..(47): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (52)..(52): Modified Deoxyribose uracil in which the 5th carbon position is substituted with a hydrophobic group of a naphthylmethylcarboxamide

SEQ ID NO: 73 - DNA, 52 bp, Artificial Sequence; identical to SEQ ID NO: 61
  A62 SSD (HEG3)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): It is substituted with methoxy at the second carbon position
  misc_binding (26)..(27): The two bases are linked each other with (Spacer Phosphoramidite 18)3
  misc_feature (30)..(30): Modifications in which fluorine is introduced at the second carbon position
  misc_feature (31)..(32): Modified Deoxyribose uracil in which the 5th carbon position is substituted with a hydrophobic group of a naphthylmethylcarboxamide
  misc_feature (34)..(34): Modifications in which fluorine is introduced at the second carbon position
  misc_feature (36)..(36): Modifications in which fluorine is introduced at the second carbon position
  misc_feature (38)..(38): Modified Deoxyribose uracil in which the 5th carbon position is substituted with a hydrophobic group of a naphthylmethylcarboxamide
  misc_feature (39)..(39): Modifications in which methoxy is introduced at the second carbon position
  misc_feature (40)..(40): Modifications in which fluorine is introduced at the second carbon position
  misc_feature (41)..(41): Modifications in which methoxy is introduced at the second carbon position
  misc_feature (42)..(42): Modified Deoxyribose uracil in which the 5th carbon position is substituted with a hydrophobic group of a naphthylmethylcarboxamide
  misc_feature (44)..(44): Modified Deoxyribose uracil in which the 5th carbon position is substituted with a hydrophobic group of a naphthylmethylcarboxamide
  misc_feature (47)..(47): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (52)..(52): Modified Deoxyribose uracil in which the 5th carbon position is substituted with a hydrophobic group of a naphthylmethylcarboxamide

SEQ ID NO: 74 - DNA, 52 bp, Artificial Sequence; identical to SEQ ID NO: 61
  A62 SSD (HEG4)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): It is substituted with methoxy at the second carbon position
  misc_binding (26)..(27): The two bases are linked each other with (Spacer Phosphoramidite 18)4
  misc_feature (30)..(30): Modifications in which fluorine is introduced at the second carbon position
  misc_feature (31)..(32): Modified Deoxyribose uracil in which the 5th carbon position is substituted with a hydrophobic group of a naphthylmethylcarboxamide
  misc_feature (34)..(34): Modifications in which fluorine is introduced at the second carbon position
  misc_feature (36)..(36): Modifications in which fluorine is introduced at the second carbon position
  misc_feature (38)..(38): Modified Deoxyribose uracil in which the 5th carbon position is substituted with a hydrophobic group of a naphthylmethylcarboxamide
  misc_feature (39)..(39): Modifications in which methoxy is introduced at the second carbon position
  misc_feature (40)..(40): Modifications in which fluorine is introduced at the second carbon position
  misc_feature (41)..(41): Modifications in which methoxy is introduced at the second carbon position
  misc_feature (42)..(42): Modified Deoxyribose uracil in which the 5th carbon position is substituted with a hydrophobic group of a naphthylmethylcarboxamide
  misc_feature (44)..(44): Modified Deoxyribose uracil in which the 5th carbon position is substituted with a hydrophobic group of a naphthylmethylcarboxamide
  misc_feature (47)..(47): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (52)..(52): Modified Deoxyribose uracil in which the 5th carbon position is substituted with a hydrophobic group of a naphthylmethylcarboxamide

SEQ ID NO: 75 - DNA, 52 bp, Artificial Sequence; identical to SEQ ID NO: 61
  A62 SSD (HEG5)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): It is substituted with methoxy at the second carbon position
  misc_binding (26)..(27): The two bases are linked each other with (Spacer Phosphoramidite 18)5
  misc_binding (30)..(30): Modifications in which fluorine is introduced at the second carbon position
  misc_binding (31)..(32): Modified Deoxyribose uracil in which the 5th carbon position is substituted with a hydrophobic group of a naphthylmethylcarboxamide
  misc_feature (31)..(32): n is a, c, g, or t
  misc_binding (34)..(34): Modifications in which fluorine is introduced at the second carbon position
  misc_binding (36)..(36): Modifications in which fluorine is introduced at the second carbon position
  misc_binding (38)..(38): Modified Deoxyribose uracil in which the 5th carbon position is substituted with a hydrophobic group of a naphthylmethylcarboxamide
  misc_feature (38)..(38): n is a, c, g, or t
  misc_binding (39)..(39): Modifications in which methoxy is introduced at the second carbon position
  misc_binding (40)..(40): Modifications in which fluorine is introduced at the second carbon position
  misc_binding (41)..(41): Modifications in which methoxy is introduced at the second carbon position
  misc_binding (42)..(42): Modified Deoxyribose uracil in which the 5th carbon position is substituted with a hydrophobic group of a naphthylmethylcarboxamide
  misc_feature (42)..(42): n is a, c, g, or t
  misc_binding (44)..(44): Modified Deoxyribose uracil in which the 5th carbon position is substituted with a hydrophobic group of a naphthylmethylcarboxamide
  misc_feature (44)..(44): n is a, c, g, or t
  misc_binding (47)..(47): Modifications in which fluorine is introduced at the second carbon position
  misc_binding (48)..(48): Modified Deoxyribose uracil in which the 5th carbon position is substituted with a hydrophobic group of a naphthylmethylcarboxamide
  misc_feature (48)..(48): n is a, c, g, or t
  misc_binding (49)..(49): Modifications in which methoxy is introduced at the second carbon position
  misc_binding (50)..(50): Modifications in which fluorine is introduced at the second carbon position
  misc_binding (52)..(52): Modified Deoxyribose uracil in which the 5th carbon position is substituted with a hydrophobic group of a naphthylmethylcarboxamide
  misc_feature (52)..(52): n is a, c, g, or t

SEQ ID NO: 76 - DNA, 52 bp, Artificial Sequence; identical to SEQ ID NO: 61
  A62 SSD (HEG6)
  misc_feature (2)..(2): Modifications in which fluorine is introduced at the second carbon position
  misc_feature (3)..(4): Modified Deoxyribose uracil in which the 5th carbon position is substituted with a hydrophobic group of a naphthylmethylcarboxamide
  misc_feature (6)..(6): Modifications in which fluorine is introduced at the second carbon position
  misc_feature (8)..(8): Modifications in which fluorine is introduced at the second carbon position
  misc_feature (10)..(10): Modified Deoxyribose uracil in which the 5th carbon position is substituted with a hydrophobic group of a naphthylmethylcarboxamide
  misc_feature (11)..(11): Modifications in which methoxy is introduced at the second carbon position
  misc_feature (12)..(12): Modifications in which fluorine is introduced at the second carbon position
  misc_feature (13)..(13): Modifications in which methoxy is introduced at the second carbon position
  misc_feature (14)..(14): Modified Deoxyribose uracil in which the 5th carbon position is substituted with a hydrophobic group of a naphthylmethylcarboxamide
  misc_feature (16)..(16): Modified Deoxyribose uracil in which the 5th carbon position is substituted with a hydrophobic group of a naphthylmethylcarboxamide
  misc_feature (19)..(19): Modifications in which fluorine is introduced at the second carbon position
  misc_feature (20)..(20): Modified Deoxyribose uracil in which the 5th carbon position is substituted with a hydrophobic group of a naphthylmethylcarboxamide
  misc_feature (21)..(21): Modifications in which methoxy is introduced at the second carbon position
  misc_feature (22)..(22): Modifications in which fluorine is introduced at the second carbon position
  misc_feature (24)..(24): Modified Deoxyribose uracil in which the 5th carbon position is substituted with a hydrophobic group of a naphthylmethylcarboxamide
  misc_feature (25)..(28): It is substituted with methoxy at the second carbon position
  misc_binding (26)..(27): The two bases are linked each other with (Spacer Phosphoramidite 18)6
  misc_feature (30)..(30): Modifications in which fluorine is introduced at the second carbon position
  misc_feature (31)..(32): Modified Deoxyribose uracil in which the 5th carbon position is substituted with a hydrophobic group of a naphthylmethylcarboxamide
  misc_feature (34)..(34): Modifications in which fluorine is introduced at the second carbon position
  misc_feature (36)..(36): Modifications in which fluorine is introduced at the second carbon position
  misc_feature (38)..(38): Modified Deoxyribose uracil in which the 5th carbon position is substituted with a hydrophobic group of a naphthylmethylcarboxamide
  misc_feature (39)..(39): Modifications in which methoxy is introduced at the second carbon position
  misc_feature (40)..(40): Modifications in which fluorine is introduced at the second carbon position
  misc_feature (41)..(41): Modifications in which methoxy is introduced at the second carbon position
  misc_feature (42)..(42): Modified Deoxyribose uracil in which the 5th carbon position is substituted with a hydrophobic group of a naphthylmethylcarboxamide
  misc_feature (44)..(44): Modified Deoxyribose uracil in which the 5th carbon position is substituted with a hydrophobic group of a naphthylmethylcarboxamide
  misc_feature (47)..(47): Modifications in which fluorine is introduced at the second carbon position
  misc_feature (48)..(48): Modified Deoxyribose uracil in which the 5th carbon position is substituted with a hydrophobic group of a naphthylmethylcarboxamide
  misc_feature (49)..(49): Modifications in which methoxy is introduced at the second carbon position
  misc_feature (50)..(50): Modifications in which fluorine is introduced at the second carbon position
  misc_feature (52)..(52): Modified Deoxyribose uracil in which the 5th carbon position is substituted with a hydrophobic group of a naphthylmethylcarboxamide

(19) United States
(12) Patent Application Publication
(10) Pub. No.: US 2025/0223600 A1
(43) Pub. Date: Jul. 10, 2025

(54) INSULIN RECEPTOR-BINDING APTAMER DIMER AND USE THEREOF

(71) Applicant: POSTECH Research and Business Development Foundation, Pohang-si, Gyeongsangbuk-do (KR)

(72) Inventors: Sung Ho RYU, Pohang-si (KR); Eun Ju OH, Pohang-si (KR); Na Oh YUNN, Pohang-si (KR); Ji Min LEE, Incheon (KR); Man Geun PARK, Daegu (KR); Seong Eun PARK, Pohang-si (KR)

(73) Assignee: POSTECH Research and Business Development Foundation, Pohang-si, Gyeongsangbuk-do (KR)

(21) Appl. No.: 18/837,461

(22) PCT Filed: Jun. 30, 2022
(86) PCT No.: PCT/KR2022/009443

§ 371 Date: Aug. 9, 2024

(51) Int. Cl. C12N 15/115; A61K 31/7088

(52) U.S. Cl. CPC C12N 15/115; A61K 31/7088; C12N 2310/16; C12N 2310/321; C12N 2310/322; C12N 2310/335; C12N 2310/51

(57) ABSTRACT

The present disclosure relates to an insulin receptor-binding aptamer dimer and uses thereof. The insulin receptor-binding aptamer dimer according to the present disclosure has a greater effect in activating insulin receptors compared to aptamer monomers, displays different phosphorylation characteristics (increased phosphorylation sites) towards insulin receptors, and particularly manages to elevate pAKT levels while not raising pERK levels. This leads to minimizing side effects that occur during long-term insulin therapy and enhancing glucose uptake efficacy, thereby exhibiting therapeutic effects. Consequently, the aptamer dimer shows excellent effects in treating diseases such as diabetes, etc.

### CROSS REFERENCE TO RELATED APPLICATIONS

This application is a National Stage of International Application No. PCT/KR2022/009443 filed on Jun. 30, 2022.

### INCORPORATION BY REFERENCE OF SEQUENCE LISTING

The content of the electronically submitted sequence listing, file name: Q301123_sequence listing as filed; size: 175, 615 bytes; and date of creation: Aug. 9, 2024, filed herewith, is incorporated herein by reference in its entirety.

### TECHNICAL FIELD

The present disclosure relates to an insulin receptor-binding aptamer dimer and use thereof.

### BACKGROUND ART

The insulin receptor (IR) is a tyrosine kinase receptor, a transmembrane receptor that is activated by insulin, IGF-1, or IGF-2. It comprises a tetrameric structure composed of two a chains (719 residues), which contain the insulin-binding portion, and two β chains (620 residues), which contain the transmembrane portion, linked by an S—S bond. When insulin binds to the receptor, tyrosine kinase activity in the intracellular region of the β-chain is activated, leading to phosphorylation of the receptor tyrosine residues. This self-phosphorylation leads to the phosphorylation of other proteins.

Insulin binding to the insulin receptor (IR) initiates a signal transduction cascade that plays an essential role in glucose homeostasis. A key step in addressing diabetes involves understanding ligand-dependent IR signaling and developing novel pharmacologic agents that modulate IR. However, despite extensive efforts over decades, the molecular mechanisms of IR activation by insulin binding are not clearly understood.

IR, a member of the receptor tyrosine kinase superfamily, is a glycoprotein composed of two α and two β subunits (α2β2) covalently linked by disulfide bonds. The extracellular domain (also referred to as the ectodomain) of the IR comprises two α subunits and the N-terminal fragment of the two β subunits, while the transmembrane domain and cytoplasmic tyrosine kinase (TK) domain comprise the C-terminal fragment of the β subunit. The insulin-binding determinant is located entirely within an ectodomain, which consists of leucine-rich repeat domains L1 and L2 of the α chain, an intervening cysteine-rich (CR) domain, and three fibronectin type III domains, Fn0, Fn1, and Fn2.

Insulin itself was the first peptide hormone to be structurally elucidated by X-ray crystallography, and has been the subject of extensive structural investigations over the past 50 years, but the molecular mechanism of IR activation by insulin remains unelucidated. There are two insulin binding sites on the IR, site 1 and 2, wherein each site 1 on one monomer of IR is close to site 2′ on the second monomer, and binding of insulin to site 1 induces its subsequent binding to site 2′, which causes a conformational change of the IR ectodomain, thus suggesting that the distance between the two intercellular TK domains is reduced, facilitating autophosphorylation.

Today, many different types of insulin derivatives have been developed and used to treat diabetes. Since insulin is the most effective hypoglycemic agent, it is widely used to normalize blood glucose in diabetic patients, but it is known that insulin administered in high concentrations for a long period of time activates cell division, which increases the incidence of cancer due to the growth of cancer cells and worsens diabetic complications such as atherosclerosis due to the growth of vascular smooth muscle cells. To solve this problem, artificial ligands using antibodies, peptides, etc., have been developed, but further technological development is still required due to low activity and unstable pharmacokinetics in vivo.

On the other hand, a lot of research and development on aptamers has been focused on functionally inhibitory aptamers. However, in recent years, there has been a growing belief that aptamer-protein binding, if it is capable of inducing appropriate structural changes in proteins, holds potential for activating protein function, and as a result, active research and development efforts are underway in this area. However, owing to the inherent characteristics of aptamers, challenges arise in their administration in humans, such as rapid degradation in the body and requirement for large doses, indicating that improvements in these areas are essential.

Against this background, the present inventors prepared a novel insulin receptor-binding aptamer dimer, which significantly increased the activity of the aptamer. In addition, the present inventors sought to minimize phosphorylation of ERK, which promotes cell division, while maintaining as much of the same hypoglycemic effect as insulin. As a result, the present disclosure was completed by developing a novel dimer that minimizes the side effects of the drug.

### DISCLOSURE

### Technical Problem

An object of the present disclosure is to provide an aptamer dimer comprising: (1) a first aptamer comprising: a sequence that specifically binds to an extracellular domain of an insulin receptor comprising the base sequence of SEQ ID NO: 1; and a linker sequence having a sequence complementary to a linker sequence of a second aptamer linked thereto, and (2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto.

Another object of the present disclosure is to provide an aptamer dimer comprising: a first aptamer sequence that specifically binds to an extracellular domain of an insulin receptor comprising the base sequence of SEQ ID NO: 1; a spacer sequence; and a second aptamer sequence that specifically binds to the extracellular domain of the insulin receptor comprising the base sequence of SEQ ID NO: 1.

Still another object of the present disclosure is to provide use of the aptamer dimer.

### Technical Solution

In one general aspect, there is provided an aptamer dimer comprising: (1) a first aptamer comprising: a sequence that specifically binds to an extracellular domain of an insulin receptor comprising the base sequence of SEQ ID NO: 1; and a linker sequence having a sequence complementary to a linker sequence of a second aptamer linked thereto, and (2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto.

In another general aspect, there is provided an aptamer dimer comprising: the first aptamer sequence that specifically binds to an extracellular domain of an insulin receptor comprising the base sequence of SEQ ID NO: 1; a spacer sequence; and a second aptamer sequence that specifically binds to the extracellular domain of the insulin receptor comprising the base sequence of SEQ ID NO: 1.

The term “aptamer” is a single-stranded oligonucleotide with the length of 15-40 nucleotides that form a specific three-dimensional conformation, has a stem loop structure, and binds specifically to a particular molecule. The aptamer is a compound that is easily synthesized chemically, readily modifiable, heat-stable, and exhibits very high specificity for its target.

Aptamer sequences may be discovered by a method known as selective evolution of ligands by exponential enrichment (SELEX), and hundreds of aptamer sequences are already publicly available. The aptamers are often compared to antibodies in that they specifically bind to targets thereof, but without the immune response. Many aptamers have consistently been discovered with the capability to bind to an extensive array of target molecules, including small molecules, peptides, and even membrane proteins. Aptamers are often compared to monoclonal antibodies because of their inherent high affinity (usually at the pM level) and specificity to bind to their target molecules, and are often referred to as ‘chemical antibodies’, which renders them highly promising as alternative antibodies. The aptamers are very stable compared to antibodies. Unlike protein and antibody drugs, which cannot be stored or transported at room temperature, the aptamers can endure such conditions and maintain their functionality even after sterilization.

The term “aptamer dimer” in the present disclosure refers to a structure comprising two aptamers linked by a suitable polymeric moiety. According to the present disclosure, the aptamer dimer is a combination of two aptamers of one type characterized by specific binding to the extracellular domain of the insulin receptor via a linker having complementary sequences and/or two or more aptamers of one type characterized by specific binding to the extracellular domain of the insulin receptor via a spacer into a single strand of aptamers. These aptamer dimer function as an agonist for the insulin receptor.

In the present disclosure, the first aptamer and the second aptamer may comprise a sequence of SEQ ID NO: 1 in the configuration of the aptamer sequence and may specifically bind to the extracellular domain of the insulin receptor.

The configuration of aptamer dimers via linker or spacer sequences may be equally or more effective, without limitation on their conformations, provided that the sequence of SEQ ID NO: 1 is organized in the 5′ to 3′ direction.

Specifically, when constructing an aptamer dimer through a linker, two aptamer regions of SEQ ID NO: 1 may be included in the dimer through complementary sequences. In this case, the sequence structure due to the complementary sequence may be configured as shown in FIG. 1. In addition, when the first aptamer and the second aptamer are connected through a spacer sequence, two consecutive aptamer sequences of SEQ ID NO: 1 in the 5′-3′ direction may be included.

In other words, the manner in which each of the first and second aptamers acts is not limited to the form or manner in which they are connected by linkers or spacers.

The base sequence of SEQ ID NO: 1 may act as an aptamer capable of specifically binding to the extracellular domain of the insulin receptor, promoting phosphorylation of the insulin receptor, and binding with specific affinity to the insulin receptor similar to insulin.

The sequence of SEQ ID NO: 1, characterized by its specific binding to the insulin receptor, and may comprise a modified base in which position 5 of the deoxyribose uracil is substituted with a hydrophobic functional group.

The bases used in the aptamers of the present disclosure, other than the above modified bases, are selected from the group consisting of bases A, G, C, T, and deoxy forms thereof, unless otherwise noted, and the inclusion of modifications thereof is specifically noted.

To increase the binding affinity and specificity of the aptamer, a position 5 of the nucleotide base within the aptamer may be substituted to a hydrophobic functional group. As an example, deoxyribose uracil modified by substituting a hydrophobic functional group at position 5 of the thymine base in the variable region, such as 5-[N-(1-naphthylmethyl)carboxamide]-2′-deoxyuridine (NapdU) or 5-(N-benzylcarboxamide)-2′-deoxyuridine, is included in the variable region. For example, the number thereof is, but not limited to, 1, 2, 3, 4, 5, 6 7, 8, 9, 10, and preferably 5, 6, 7, 8, or 9. The hydrophobic functional group may comprise a naphthyl group, a benzyl group, a pyrrolebenzyl group, an isobutyl group, or a tryptophan, and more preferably the hydrophobic functional group is a naphthyl group or a benzyl group. Deoxyribose uracil modified by substitution with a naphthyl group is shown in Chemical Formula 1 below, and deoxyribose uracil modified by substitution with a benzyl group is shown in Chemical Formula 2 below:

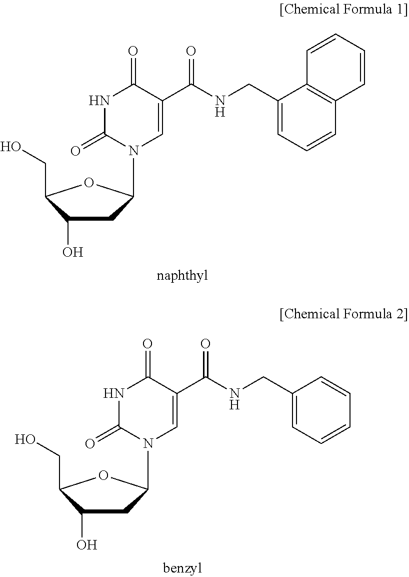

In the present disclosure, the base sequence of SEQ ID NO: 1 (IR-A62) is as follows:

SEQ ID NO: 1 CANNACGCAN GAGNCNAGAN CCGN

In the above sequence, N may be a modified base, with a naphthyl group or a benzyl group as a hydrophobic functional group, as referred to in Chemical Formula 1 or 2 above.

Specifically, the N group may be deoxyribose uracil with a hydrophobic functional group substituted at the 5th carbon position. More specifically, the N group may be at least any one selected from the group consisting of naphthyl-uracil nucleotide (5-(N-1-naphthylmethylcarboxamide)-2′-deoxyuridine, Naphthyl-dU, NapdU) or benzyl-uracil nucleotide (5-(N-benzylcarboxamide)-2′-deoxyuridine, Benzyl-dU, BndU).

The above-described bases C, A, and G may be modified or unmodified bases.

For example, the modified C may include a modification of an introduction of a methoxy group (2-O-Me, 2-O-Methoxy-DNA) at the C2 position of the nucleotide; or an introduction of fluorine (2-F, 2-Fluorine-DNA) at the C2 position of the nucleotide.

Specifically, the modified G may include a modification of an introduction of a methoxy group (2-O-Me, 2-O-Methoxy-DNA) at the C2 position of the nucleotide; or an introduction of fluorine (2-F, 2-Fluorine-DNA) at the C2 position of the nucleotide.

Specifically, the modified A may include a modification of an introduction of a methoxy group (2-O-Me, 2-O-Methoxy-DNA) at the C2 position of the nucleotide; or an introduction of fluorine (2-F, 2-Fluorine-DNA) at the C2 position of the nucleotide.

SEQ ID NO: 1 corresponds to 22nd to 46th sequences in the base sequence of SEQ ID NO: 2.

SEQ ID NO: 2
TATGAGTGAC CGTCCGCCTG GCANNACGCA NGAGNCNAGA
NCCGNCAGAC CNAAGGCNNC AGCCACACCA CCAGCCAAA

The base sequence of SEQ ID NO: 1 may form a stem-loop structure comprising nucleotides inside. The stem-loop structure may form one or two stem-loop structures. The base sequence in SEQ ID NO: 1 corresponds to the sequence essential to form the stem-loop structure, which is characterized by its specific binding to the insulin receptor.

Thus, in the present disclosure, the first aptamer and the second aptamer essentially comprise the base sequence of SEQ ID NO: 1. More specifically, SEQ ID NO: 1 may further comprise 25 to 79 consecutive base sequences, comprising 22nd to 46th in the base sequence of SEQ ID NO: 2. Specifically, the SEQ ID NO: 1 may be a sequence of 25, 26, 27, 28, 29, 30, 35, 40, 45, 50, 60, 70, or 79 consecutive sequences, comprising 22nd to 46th of SEQ ID NO: 2.

In the present disclosure, comprising a sequence may mean including a sequence, may mean essentially consisting of a sequence, or may mean consisting of a sequence.

Furthermore, in any base sequence other than the position of N in SEQ ID NO: 1 and/or SEQ ID NO: 2, at least one base located at the 5′ end, 3′ end, middle or both ends may be modified to enhance stability in serum or to modulate renal clearance. The modification may be achieved by one or more selected from the group consisting of polyethylene glycol (PEG), biotin, inverted deoxythymidine (idT), locked nucleic acid (LNA), 2′-methoxy nucleoside, 2′-amino nucleoside, 2′F-nucleoside, amine linker, thiol linker, and cholesterol bound at the 5′ end, 3′ end, middle, or both ends. The 2′-methoxy nucleoside, 2′-amino nucleoside, or 2′F-nucleoside binds to a base within the aptamer to provide a modified base to confer nuclease resistance.

Preferably, a modified base may be further included to secure nuclease resistance and provide stability when administered in humans. The modified base may preferably be a 2′-OMe (methoxy) and/or 2′-F (fluorine) substitution on any base A, C, T, or G. In other words, at least one 2′-OMe (methoxy) or 2′-F (fluorine) modified base substitution on the base A, C, T, or G of SEQ ID NO: 1 and/or SEQ ID NO: 2 above, may be included.

For better understanding of 41 the above base modifications, examples of any modified sequences centered on the base sequence of SEQ ID NO: 1 are listed in SEQ ID NO. 3 to 11 below, but these sequences are provided as exemplary sequences of the present disclosure and are not intended to impose limitations.

TABLE 1

| | | | Hydrophobic | |
|---|---|---|---|---|
| | | | functional | |
| | | | group of | Modifi- |
| SEQ | Sequence | Length | modified | cation |
| ID NO | (5′ → 3′) | (mer) | base N | of base |
| 3 | GCCTGGCANN | 30 | Naphthyl | None |
| | ACGCANGAGN | | group | |
| | CNAGANCCGN | | | |
| | CAGACCNAA | | | |
| 4 | CANNACGCAN | 27 | Naphthyl | 2′-OMe |
| | GAGNCNAGAN | | group/ | 2′-F |
| | CCGNCAG | | Benzyl | |
| | | | group | |
| 5 | CANNACGCAN | 26 | Naphthyl | 2′-OMe |
| | GAGNCNAGAN | | group/ | 2′-F |
| | CCGNCA | | Benzyl | |
| | | | group | |
| 6 | CANNACGCAN | 26 | Naphthyl | 2′-OMe |
| | GAGNCNAGAN | | group/ | 2′-F |
| | CCGNC | | Benzyl | |
| | | | group | |
| 7 | CANNACGCAN | 24 | Naphthyl | 2′-OMe |
| | GAGNCNAGAN | | group | 2′-F |
| | CCGN | | | |
| 8 | CANNACGCAN | 27 | Naphthyl | 2-OMe |
| | GAGNCNAGAN | | group/ | 2′-F |
| | CCGNCAG | | Benzyl | |
| | | | group | |
| 9 | CANNACGCAN | 26 | Naphthyl | 2′-OMe |
| | GAGNCNAGAN | | group/ | 2′-F |
| | CCGNCA | | Benzyl | |
| | | | group | |
| 10 | CANNACGCAN | 27 | Naphthyl | 2′-OMe |
| | GAGNCNAGAN | | group/ | 2′-F |
| | CCGNCAG | | Benzyl | |
| | | | group | |
| 11 | CANNACGCAN | 27 | Naphthyl | 2′-OMe |
| | GAGNCNAGAN | | group/ | 2′-F |
| | CCGNCAG | | Benzyl | |
| | | | group | |

Bold and underlined N: Chemical Formula 2 hydrophobic functional group (benzylcarboxamide)
Bold and italicized N: Chemical Formula 1 hydrophobic functional group (naphthylmethylcarboxamide)
Underlined A, G, and C: base modifications with fluoro (2′-F) at 2′ position
Italicized A, G, and C: base modifications with methoxy (2′-Ome) at 2′ position

Accordingly, any exemplary embodiment according to the present disclosure may comprise a modification configuration comprising any of the above-described sequences.

For example, it may be an aptamer dimer comprising: (1) a first aptamer comprising: a sequence that specifically binds to an extracellular domain of an insulin receptor comprising SEQ ID NO: 1, preferably any one base sequence selected from the group consisting of SEQ ID NO. 3 to 11; and a linker sequence having a sequence complementary to a linker sequence of a second aptamer linked thereto, and (2) the second aptamer comprising: a sequence that specifically binds to the extracellular domain of the insulin receptor comprising SEQ ID NO: 1, preferably any one base sequence selected from the group consisting of SEQ ID NO. 3 to 11; and a linker sequence having a sequence complementary to the linker sequence of the first aptamer linked thereto.

Further, there is provided an aptamer dimer comprising: a first aptamer sequence that specifically binds to an extracellular domain of an insulin receptor comprising the base sequence of SEQ ID NO: 1, preferably any one base sequence selected from the group consisting of SEQ ID NO. 3 to 11; a spacer sequence; and a second aptamer sequence that specifically binds to the extracellular domain of the insulin receptor comprising SEQ ID NO: 1, preferably any one base sequence selected from the group consisting of SEQ ID NO. 3 to 11.

As described above, the structure for increasing the binding force and specificity according to the present disclosure, any modifications to the base sequence of SEQ ID NO: 1 to improve stability in serum, or to modulate renal clearance are included within the scope of the present disclosure.

The number of the modified bases may be from 4 to 60, preferably from 6 to 50.

When the aptamer dimer according to the present disclosure binds to the insulin receptor, the receptor tyrosine residues are phosphorylated, and the phosphorylation of the insulin receptor increases glucose uptake by regulating the transportation of glucose transporter 4 (GLUT4) in adipocytes and muscle through signaling. The metabolic functions via these insulin receptors are mainly regulated by the IRS-AKT pathway, and in addition, phosphoinositide 3-kinase (PI3K) plays an important role in the phosphorylation of protein kinase B (AKT) during insulin signaling.

A single aptamer comprising the base sequence of SEQ ID NO: 1 alone has the ability to bind to the insulin receptor, but it does not sufficiently mimic the function of insulin and is less effective than insulin, necessitating high doses for administration in the human body.

Therefore, it was confirmed in the present disclosure that when a dimer centered on the base sequence of SEQ ID NO: 1 is prepared and administered, it mimics the function of insulin almost identically and does not affect the ERK phosphorylation pathway, thus enabling precise blood glucose control without side effects such as an increase in the incidence of cancer due to the growth of cancer cells that may occur due to the promotion of cell division and the aggravation of diabetic complications such as atherosclerosis due to the growth of vascular smooth muscle cells.

In particular, in order to achieve a suitable structure as a dimer, it is possible to show excellent behavior by connecting the aptamers via a linker sequence through a complementary sequence. Specifically, compared to dimers composed of nucleic acid materials or single-stranded dimers, the dimer linked by the complementary sequence of the present disclosure have a shorter synthesis length, resulting in higher purity and synthesis efficiency in large-scale synthesis, and lower synthesis costs.

Accordingly, the first aptamer according to the present disclosure comprises a linker sequence having a sequence complementary to the linker sequence of the second aptamer linked to the sequence that specifically binds to the extracellular domain of the insulin receptor comprising the base sequence of SEQ ID NO: 1. The second aptamer comprises a linker sequence having a sequence complementary to the linker sequence of the first aptamer linked to the sequence that specifically binds to the extracellular domain of the insulin receptor comprising the base sequence of SEQ ID NO: 1.

In the present disclosure, the first aptamer may preferably be any one selected from the group consisting of SEQ ID NO: 28, 30, 32, 34, 36, 38, 40 and 42.

In the present disclosure, the second aptamer may preferably be any one selected from the group consisting of SEQ ID NO: 29, 31, 33, 35, 37, 39, 41, and 43.

In the present disclosure, the aptamer dimer may preferably comprise any one of the first aptamer and the second aptamer selected from the group consisting of SEQ ID NOs: 28 and 29, 30 and 31, 32 and 33, 34 and 35, 36 and 37, 38 and 39, 40 and 41, and 42 and 43.

In the present disclosure, the formation of a dimer may be implemented by binding two aptamers via the complementary nucleic acid sequences to achieve a dimer form and/or by binding two aptamers via the spacer sequence to achieve a single-stranded form.

The term “linker sequence” refers to a chemical moiety that connects two molecules or moieties. The linker sequence of the present disclosure corresponds to a nucleic acid linker, which is linked to the first aptamer and the second aptamer to form a double bond through their complementary base sequences, thereby forming a dimer.

An example of the dimeric form is shown in FIG. 1. As can be seen in FIG. 1, the dimer form may be obtained in which the first aptamer and second aptamer are linked through the binding of complementary linker sequences.

The first aptamer and the second aptamer comprising a sequence of SEQ ID No: 1 that specifically binds to the extracellular domain of the insulin receptor via the linker having the complementary sequence may have the same active site sequence or may have different sequences. In other words, sequences that specifically bind to the extracellular domain of the insulin receptor in two aptamers may be the same or different.

The linker sequence of the linker having the above complementary sequence may have, for example, 1, 2, 3, 4, 5, 6, 7, 8, 9, 10, 15, 20, 25, 30, 40, 50, 60 sequences, preferably have 5 to 30 base sequences, 10 to 30 base sequences, approximately 15 to 25 base sequences, and more preferably about 20 base sequences.

The linker sequence may further comprise a modified base to secure nuclease resistance and provide stability when administered in humans. The modified base may preferably be a 2′-OMe (methoxy) and/or 2′-F (fluorine) substitution on any base A, C, T, or G, and may be L-form DNA or LNA (Locked DNA). In other words, the base sequence of the linker sequence may have at least one 2′-OMe (methoxy) or 2′-F (fluorine) modified base substitution on the base A, C, T, or G, and may utilize a form of L-form DNA or Locked DNA (LNA) within the linker sequence.

The linker sequence of the present disclosure may preferably comprise any one sequence selected from the group consisting of SEQ ID NO: 12 to 27.

Further, if desired, it may further comprise a spacer sequence to provide appropriate spacing between the linker sequence and the sequence of SEQ ID No: 1 that binds to the insulin receptor. The sequence for providing space between the linker and the insulin receptor forming these complementary binding is preferably polyT and may be included between the linker and the insulin receptor at approximately 0, 1, 2, 3, 4, 5, or 6 fragments.

Thus, the first aptamer may further comprise a spacer sequence between the linker sequence and the sequence that specifically binds to the extracellular domain of the insulin receptor comprising the base sequence of SEQ ID NO: 1. The second aptamer may further comprise a spacer sequence between the linker sequence and the sequence that specifically binds to the extracellular domain of the insulin receptor comprising the base sequence of SEQ ID NO: 1.

Further, in the present disclosure, the spacer sequence may be utilized as a binding material to combine two kinds of aptamers and/or two or more of one kind aptamer to produce a single strand.

In other words, the present disclosure provides the aptamer dimer comprising: the first aptamer sequence that specifically binds to an extracellular domain of an insulin receptor comprising the base sequence of SEQ ID NO: 1; the spacer sequence; and the second aptamer sequence that specifically binds to the extracellular domain of the insulin receptor comprising the base sequence of SEQ ID NO: 1.

Specifically, the aptamer dimer of the present disclosure may comprise: the first aptamer sequence that specifically binds to an extracellular domain of an insulin receptor comprising the base sequence of SEQ ID NO: 1; the spacer sequence; and the second aptamer sequence that specifically binds to the extracellular domain of the insulin receptor comprising the base sequence of SEQ ID NO: 1.

The spacer sequences are preferably poly A fragments, poly T fragments, poly G fragments, poly C fragments, phosphoramidite fragments, random oligonucleotide sequences that do not interfere with the aptamer sequence, and ethylene glycol fragments, the length of which may vary as known to those skilled in the art. Preferably, the spacer sequence may be 2, 3, 4, 5, 6, 7, 8, 9, 10, 11, 12, 13, 14, 15, 16, 17, 18, 19, 20, 21, 22, 23, 24, 25, 26, 27, 28, 29, or 30 poly T fragment sequences.

The spacer sequence may further comprise a modified base to secure nuclease resistance and provide stability when administered in humans. The modified base may preferably be a 2′-OMe (methoxy) and/or 2′-F (fluorine) substitution on any base A, C, T, or G, and may be L-form DNA or LNA (Locked DNA). In other words, the sequence of the linker sequence may have at least one 2′-OMe (methoxy) or 2′-F (fluorine) modified base substitution on the base A, C, T, or G, and may utilize a form of L-form DNA or Locked DNA (LNA) within the linker sequence.

The linker sequence of the present disclosure may preferably comprise any one sequence selected from the group consisting of SEQ ID NO: 46 to 60.

Accordingly, the present disclosure provides the aptamer dimer comprising: (1) the first aptamer comprising: a sequence that specifically binds to an extracellular domain of an insulin receptor comprising the base sequence of SEQ ID NO: 1; and a linker sequence having sequence complementary to a linker sequence of a second aptamer linked thereto, and (2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto.

In addition, the present disclosure provides the aptamer dimer comprising: the first aptamer sequence that specifically binds to the extracellular domain of the insulin receptor comprising the base sequence of SEQ ID NO: 1; the spacer sequence; and the second aptamer sequence that specifically binds to the extracellular domain of the insulin receptor comprising the base sequence of SEQ ID NO: 1.

In the present disclosure, the aptamer dimer may preferably be any one selected from the group consisting of SEQ ID NO: 61 to 76.

The aptamer dimer of the present disclosure may exhibit effects that may more closely mimic the action of insulin compared to the monomer form. Specifically, the aptamer dimer of the present disclosure significantly activates insulin receptors, and this action is similar to the level of action of insulin. Further, by elevating pAKT levels while not raising pERK levels, the therapeutic effect may be achieved while minimizing the side effects of long-term insulin therapy.

According to an embodiment of the present disclosure, the aptamer dimer according to the present disclosure may exhibit excellent good phosphorylation effect on Y960, Y1146, Y1150, Y1151, Y1316, and Y1322 of the insulin receptor, which is very similar to that of insulin.

According to an embodiment of the present disclosure, the aptamer dimer according to the present disclosure may exhibit superior phosphorylation effect on Y896 of the insulin receptor substrate compared to aptamer monomers, which is very similar to that of insulin.

According to an embodiment of the present disclosure, the aptamer dimer according to the present disclosure may exhibit superior phosphorylation effect on AKT (S473 as an example) compared to aptamer monomers, which is very similar to that of insulin.

According to an aspect of the present disclosure, the aptamer dimer according to the present disclosure does not exhibit phosphorylation effect on ERK relative to aptamer monomers. Accordingly, it is possible to minimize side effects caused by ERK activity that occur during long-term insulin therapy.

According to an embodiment of the present disclosure, the aptamer dimer according to the present disclosure increases glucose uptake relative to aptamer monomers, which is very similar to insulin.

In addition, the excellent serum stability due to dimer formation may minimize the half-life issues that aptamers may have in humans, maximizing the possibility of use as a therapeutic agent.

As another aspect for achieving the above object, the present disclosure provides a pharmaceutical composition comprising the aptamer dimer comprising: a first aptamer comprising: a sequence that specifically binds to an extracellular domain of an insulin receptor comprising the base sequence of SEQ ID NO: 1; and a linker sequence having a sequence complementary to a linker sequence of a second aptamer linked thereto, and the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto.

In addition, the present disclosure provides a pharmaceutical composition comprising the aptamer dimer comprising: the first aptamer sequence that specifically binds to an extracellular domain of an insulin receptor comprising the base sequence of SEQ ID NO: 1; the spacer sequence; and the second aptamer sequence that specifically binds to the extracellular domain of the insulin receptor comprising the base sequence of SEQ ID NO: 1.

As still another aspect for achieving the above object, the present disclosure provides a pharmaceutical composition comprising (1) the aptamer dimer comprising: a first aptamer comprising: a sequence that specifically binds to an extracellular domain of an insulin receptor comprising the base sequence of SEQ ID NO: 1; and a linker sequence having a sequence complementary to a linker sequence of a second aptamer linked thereto, and (2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto; and a pharmaceutically acceptable carrier.

In addition, the present disclosure provides a pharmaceutical composition comprising the aptamer dimer comprising: the first aptamer sequence that specifically binds to an extracellular domain of an insulin receptor comprising the base sequence of SEQ ID NO: 1; the spacer sequence; and the second aptamer sequence that specifically binds to the extracellular domain of the insulin receptor comprising the base sequence of SEQ ID NO: 1; and a pharmaceutically acceptable carrier.

As another aspect for achieving the above object, the present disclosure provides a pharmaceutical composition for preventing or treating a metabolic disease, comprising the aptamer dimer comprising: (1) a first aptamer comprising: a sequence that specifically binds to an extracellular domain of an insulin receptor comprising the base sequence of SEQ ID NO: 1; and the linker sequence having a sequence complementary to a linker sequence of a second aptamer linked thereto, and (2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto.

Further, the present disclosure provides a pharmaceutical composition for preventing or treating a metabolic disease, comprising the aptamer dimer comprising: the first aptamer sequence that specifically binds to an extracellular domain of an insulin receptor comprising the base sequence of SEQ ID NO: 1; the spacer sequence; and the second aptamer sequence that specifically binds to the extracellular domain of the insulin receptor comprising the base sequence of SEQ ID NO: 1.

As still another aspect for achieving the above object, the present disclosure provides a method of preventing or treating a metabolic disease, comprising the aptamer dimer comprising: (1) a first aptamer comprising: a sequence that specifically binds to an extracellular domain of an insulin receptor comprising the base sequence of SEQ ID NO: 1; and the linker sequence having a sequence complementary to a linker sequence of a second aptamer linked thereto, and (2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto.

Further, the present disclosure provides a method of preventing or treating a metabolic disease, comprising the aptamer dimer comprising: the first aptamer sequence that specifically binds to an extracellular domain of an insulin receptor comprising the base sequence of SEQ ID NO: 1; the spacer sequence; and the second aptamer sequence that specifically binds to the extracellular domain of the insulin receptor comprising the base sequence of SEQ ID NO: 1.

The present disclosure provides an aptamer dimer for use in the prevention or treatment of a metabolic disease, comprising: (1) the first aptamer comprising: a sequence that specifically binds to an extracellular domain of an insulin receptor comprising the base sequence of SEQ ID NO: 1; and the linker sequence having a sequence complementary to a linker sequence of a second aptamer linked thereto, and (2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto.

Further, the present disclosure provides an aptamer dimer for use in the prevention or treatment of a metabolic disease, comprising: the first aptamer sequence that specifically binds to an extracellular domain of an insulin receptor comprising the base sequence of SEQ ID NO: 1; the spacer sequence; and the second aptamer sequence that specifically binds to the extracellular domain of the insulin receptor comprising the base sequence of SEQ ID NO: 1.

The present disclosure provides use of an aptamer dimer in the manufacture of a medicament for the treatment of a metabolic disease, comprising: (1) the first aptamer comprising: a sequence that specifically binds to an extracellular domain of an insulin receptor comprising the base sequence of SEQ ID NO: 1; and the linker sequence having a sequence complementary to a linker sequence of a second aptamer linked thereto, and (2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto.

Further, the present disclosure provides use of an aptamer dimer in the manufacture of a medicament for the treatment of a metabolic disease, comprising: the first aptamer sequence that specifically binds to an extracellular domain of an insulin receptor comprising the base sequence of SEQ ID NO: 1; the spacer sequence; and the second aptamer sequence that specifically binds to the extracellular domain of the insulin receptor comprising the base sequence of SEQ ID NO: 1.

The metabolic disease may be, for example, diabetes (T1D and/or T2DM, such as prediabetes), idiopathic type 1 diabetes (T1D (type 1b)), latent autoimmune diabetes in adults (LADA), early-onset type 2 diabetes (T2DM (EOD)), young-onset atypical diabetes (YOAD), maturity-onset diabetes of the young (MODY), malnutrition-associated diabetes, gestational diabetes, hyperglycemia, insulin resistance, hepatic insulin resistance, impaired glucose tolerance, diabetic neuropathy, diabetic nephropathy, kidney diseases (such as acute kidney injury, tubular dysfunction, and proinflammatory changes to proximal tubules), diabetic retinopathy, adipocyte dysfunction, visceral fat accumulation, sleep apnea, obesity (such as hypothalamic obesity and monogenic obesity) and related comorbidities (such as osteoarthritis and urinary incontinence), eating disorders (such as binge eating syndrome, bulimia nervosa, and syndromic obesity, e.g., Prader-Willi syndrome and Bardet-Biedl syndrome), weight gain due to the use of other medicaments (e.g., the use of steroids and antipsychotics), excessive sugar cravings, dyslipidemia (including hyperlipidemia, hypertriglyceridemia, elevated total cholesterol, high LDL cholesterol, and low HDL cholesterol), and non-alcoholic fatty liver diseases (NAFLDs) (including related conditions, such as steatosis, non-alcoholic steatohepatitis (NASH), fibrosis, cirrhosis, and hepatocellular carcinoma).

The aptamer dimer according to the invention may enable glucose uptake at insulin levels.

The insulin receptor-binding aptamer dimer according to the present disclosure may perform glucose uptake as an agonist for the insulin receptor, and may regulate the level of insulin without inducing cell division or other side effects that may be caused by insulin. Thus, the insulin receptor-binding aptamer dimer according to the present disclosure may be useful for controlling blood glucose without affecting the increase in cancer incidence due to the growth of cancer cells and atherosclerosis caused by the growth of vascular smooth muscle cells.

Further, the aptamer dimer of the present disclosure may increase glucose uptake of cells while having a lower lipolysis inhibitory effect. It is preferably characterized by increasing glucose uptake while exhibiting a lower lipolysis inhibitory effect compared to insulin. This lipolysis inhibitory activity is a typical role of insulin, which is known to control blood glucose but induce weight gain when administered with insulin. The aptamer dimer according to the present disclosure may effectively increase glucose uptake despite low lipolysis inhibition and, more preferably, may be effectively employed for controlling blood glucose without affecting weight gain due to lipolysis inhibition. Accordingly, the aptamer dimer according to the present disclosure may be useful in the treatment, amelioration and prevention of the metabolic disease.

As used herein, the term “pharmaceutically effective amount” refers to an amount sufficient to treat a disease at a reasonable benefit/risk ratio applicable to medical treatment, and the effective dose level may be determined depending on factors including the patient gender, age, type and severity of disease, the activity of the drug, sensitivity to the drug, the time of administration, the route of administration, the rate of excretion, the duration of treatment, drugs used concurrently, and other factors well known in the medical field.

The pharmaceutical composition of the present disclosure may be administered as an individual therapeutic agent or in combination with other therapeutic agents, and may be administered sequentially or simultaneously with a commercially available therapeutic agent. Further, the pharmaceutical composition of the present disclosure may be administered as a single dose or in multiple doses. considering all of the above factors, it is important to administer an amount capable of obtaining the maximum effect with the minimum amount without side effects, which may be easily determined by those skilled in the art.

As used herein, the term “subject” includes an animal or human whose symptoms may be ameliorated by the administration of the pharmaceutical composition according to the present disclosure. By administering the composition for treatment according to the present disclosure to an individual, insulin dysfunction may be effectively prevented and treated.

As used herein, the term “administration” means introducing a predetermined substance into humans or non-human animals by any appropriate method, and the administration route of the composition for treatment of the present disclosure may be oral or parenteral through any general route as long as the composition is possible to reach the target tissue. Further, the composition for treatment of the present disclosure may be administered by any device capable of delivering an active ingredient to a target cell.

The preferred dosage of the pharmaceutical composition according to the present disclosure depends on the condition and weight of the patient, the severity of the disease, the form of the drug, the route of administration and the duration, but may be suitably selected by those skilled in the art.

The pharmaceutical composition may be formulated in various oral dosage forms or parenteral dosage forms. For example, the composition may be in any formulation for oral administration, such as tablets, pills, hard and soft capsules, liquids, suspensions, emulsions, syrups, granules, elixirs, and the like. Such oral dosage forms may contain, in addition to the active ingredient, pharmaceutically acceptable carriers, such as, for example, diluents such as lactose, dextrose, sucrose, mannitol, sorbitol, cellulose and/or glycine, or lubricants such as silica, talc, stearic acid, and magnesium or calcium salts thereof and/or polyethylene glycol, depending on the usual composition of each dosage form.

Further, the dosage form for oral administration may comprise, in the case of a tablet, a binder such as magnesium aluminum silicate, starch paste, gelatin, tragacanth, methylcellulose, sodium carboxymethylcellulose and/or polyvinylpyrrolidine, and in some cases, may comprise a disintegrating agent such as starch, agar, alginic acid or sodium salts thereof, or a boiling mixture and/or absorbent, colorant, flavor or sweetener.

When provided for parenteral use, the dosage form may be a liquid dosage form, such as a liquid, gel, cleansing composition, tablet for insertion, suppository form, topical administration such as cream, ointment, dressing solution, spray, and other coating agents, a solution form, a suspension form, an emulsion type, etc., and may comprise an external skin preparation such as a sterilized aqueous solution, non-aqueous solvent, suspension, emulsion, freeze-dried formulation, suppository, cream, ointment, jelly, foam, detergent or insert, preferably a liquid or gel, cleansing composition, and a tablet for insertion, etc. The formulation may be prepared, for example, by adding a solubilizing agent, emulsifier, buffer for pH adjustment, etc., to sterile water. As the non-aqueous solvent or the suspension, propylene glycol, polyethylene glycol, vegetable oils such as olive oil, and injectable esters such as ethyl oleate, etc., may be used.

Optionally, the aptamer may be entrapped within a colloidal drug delivery system (such as liposome, albumin microsphere, microemulsion, nanoparticle, and nanocapsule) or macroemulsion, e.g., within microcapsules prepared by coacervation techniques or interfacial polymerization, such as hydroxymethylcellulose or gelatin-microcapsules and poly-(methylmethacrylate) microcapsules, respectively. These techniques are known in the relevant art.

The pharmaceutical composition according to the present disclosure may be administered in a pharmaceutically effective amount. As used herein, the “pharmaceutically effective amount” means an amount sufficient to prevent or treat the diseases at a reasonable benefit/risk ratio applicable to medical treatment. The effective dose level may be varied by those skilled in the art depending on factors such as method of formulation, patient condition and weight, patient gender, age, severity of disease, drug form, route and duration of administration, rate of excretion, response sensitivity, etc. The effective amount may vary depending on the route of processing, the use of excipients and the possibility of use with other medicaments, as recognized by those skilled in the art. However, for desirable effects, in the case of oral administration, the composition of the present disclosure may be administered to an adult at an amount of 0.0001 to 100 mg/kg of body weight per day, preferably 0.001 to 100 mg/kg per day, but the above dosages are not intended to limit the scope of the present disclosure in any way.

Further, the pharmaceutical composition may be formulated in a parenteral dosage form, in which case it is administered by a parenteral route, such as subcutaneous injection, intravenous injection, intramuscular injection, or intrathoracic injection. Here, for formulation into a dosage form for parenteral administration, the pharmaceutical composition may be prepared as a solution or suspension in which the active ingredient, i.e., a derivative of Chemical Formula I or a pharmaceutically acceptable salt thereof, is mixed in water with a stabilizer or buffer, and this solution or suspension may be prepared in unit dosage form in ampoules or vials.

In addition, the pharmaceutical composition may be sterile, may further comprise an auxiliary such as preservative, stabilizer, hydrating agent or emulsification promoter, salt and/or buffer for osmotic pressure control, or may comprise other further therapeutically useful substances, and may be formulated according to conventional methods of mixing, granulating, or coating.

The pharmaceutical composition of the present disclosure may be administered to a mammal including a human, preferably a rodent or a human.

### Advantageous Effects

The insulin receptor-binding aptamer dimer according to the present disclosure has a greater effect in activating insulin receptors compared to aptamer monomers, displays different phosphorylation characteristics (increased phosphorylation sites) towards insulin receptors, and particularly manages to elevate pAKT levels while not raising pERK levels. This leads to minimizing side effects that occur during long-term insulin therapy and enhancing glucose uptake efficacy, thereby exhibiting therapeutic effects. Consequently, the aptamer dimer shows excellent effects in treating diseases such as diabetes, etc.

### DESCRIPTION OF DRAWINGS

FIG. 1 shows the conformation of the binding structure by a complementary sequence of an aptamer dimer (IR-A62) according to an embodiment of the present disclosure.

FIG. 2 shows the results of confirming phosphorylation changes of insulin receptors (Y960, Y1146, Y1150, Y1151, Y1316, and Y1322), insulin receptor substrate (Y896), AKT (S473), and ERK upon treatment of the aptamer dimer according to an embodiment of the present disclosure.

FIG. 3 (A) shows the results of native PAGE showing the formation of 6-20 bp dimer by linker length, and FIG. 3 (B) shows the results of phosphorylation changes of insulin receptors (Y1150 and Y1150/Y1151), AKT (S473 and T308), and ERK (T202/Y204) upon treatment of the aptame dimer according to an embodiment of the present disclosure.

FIG. 4 shows the results of confirming phosphorylation changes of insulin receptors (Y1150 and Y1150/Y1151) and AKT (S473) upon concentration-specific treatment with the aptamer dimer according to an embodiment of the present disclosure.

FIG. 5 shows the results of confirming changes in glucose uptake of insulin receptors upon concentration-specific treatment with the aptamer dimer according to an embodiment of the present disclosure.

FIG. 6 shows the r results of increasing the phosphorylation level of Y1150 upon concentration-specific treatment with the aptamer dimer according to an embodiment of the present disclosure.

FIG. 7 shows the results of increasing the phosphorylation level (pY) of the insulin receptor upon concentration-specific treatment with the aptamer dimer according to an embodiment of the present disclosure.

FIG. 8 shows the results of increasing the phosphorylation level of AKT upon concentration-specific treatment with the aptamer dimer according to an embodiment of the present disclosure.

FIG. shows the results of increasing the phosphorylation level of ERK (T202/Y204) upon concentration-specific treatment with the aptamer dimer according to an embodiment of the present disclosure.

FIG. 10 shows the results of increasing the phosphorylation level of ERK upon concentration-specific treatment with the aptamer dimer having a modified (2′-OMe modified) linker sequence according to an embodiment of the Insulin, A62-M: IR-A62, A62 present disclosure (Ins: LD1(2′OMe): SEQ ID NO: 19, A62 LD2(2′OMe): SEQ ID NO: 20, and A62-D(2′OMe): Self-assembly (dimer) of SEQ ID NO: 19 and SEQ ID NO: 20).

FIGS. 11(a) and 11(b) show the results of confirming the degradation rate of aptamer dimer in serum according to an embodiment of the present disclosure.

FIG. 12 shows the results of confirming phosphorylation changes of insulin receptors (Y1150/Y1151 cs and Y1150/Y1151 pAb) and AKT (S473) depending on the length of the linker of the aptamer dimer according to an embodiment of the present disclosure.

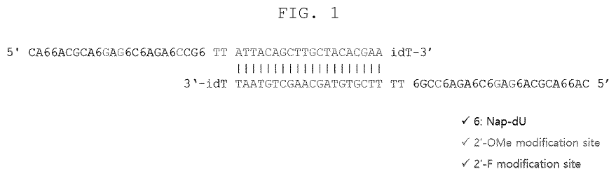

### BEST MODE

The following Examples are presented to facilitate the understanding of the present disclosure. These Examples and Preparation Examples are only provided to more easily understand the present disclosure, but the content of the present disclosure is not limited by these Examples.

### <Example 1> Synthesis of Insulin Receptor-Binding Aptamer Dimer

The aptamers were synthesized by Aptamer Science, Inc. (Pohang, Korea). Specifically, the aptamers used in the experiments were synthesized using the following methods. Aptamers were synthesized by solid phase oligo synthesis using Bioautomation's Mermade 12 synthesizer, a nucleic acid-only stationary phase synthesizer. The aptamer was synthesized with solid phase b-cyanoethyl phosphoramidite chemistry using an oligonucleotide synthesizer (Bioautomation, Mermade12), and after synthesis, CPG (200 nmole synthesis column, 1000A (MM1-1000-)) was put in a cleavage solution [t-butylamine:methanol:water (1:1:2 volume ratio)], and the product was subjected to cleavage/deprotection at 70° C. for 5 hours, followed by vacuum drying and separation/purification by HPLC (GE, AKTA basic). The column used was an RP-C18 column (Waters, Xbridge OST C18 10×50 mm) with UV 254 nm/290 nm, flow rate: 5 ml/min, temperature: 65° C., and 0.1M TEAB/Acetonitrile Buffer. All of these aptamers were determined by LC-ESI MS spectrometer (Waters HPLC systems (Waters)+Qtrap2000 (ABI)) with accurate molecular weights within 0.02% error, and had 80-90% purity when measured by HPLC.

The sequence information based on IR-A62 is shown in Table 2 below.

TABLE 2

| Name | Sequence (5′→3′) |
|---|---|
| IR-A62 ms monomer | CANNACGCAN GAGNCNAGAN CCGN-idT |
| (SEQ ID NO: 7) | |
| IR-A62 ms-LD1 | CANNACGCAN GAGNCNAGAN CCGN TT |
| (SEQ ID NO: 42) | ATTACAGCTTGCTACACGAA-idT |
| IR-A62 ms-LD2 | CANNACGCAN GAGNCNAGAN CCGN TT |
| (SEQ ID NO: 43) | TTCGTGTAGCAAGCTGTAAT-idT |

Bold and italicized N: hydrophobic functional group (naphthylmethylcarboxamide) of Chemical Formula 1
Underlined A, G, and C: base modifications with fluoro (2′-F) at 2′ position
Italicized A, G, and C: base modifications with methoxy (2′-Ome) at 2′ position

### <Example 2> Confirmation of Specific Phosphorylation of Tyrosine Residues on Insulin Receptor

To confirm the specific phosphorylation of tyrosine residues on the insulin receptor, experiments were performed with insulin [Sigma-Aldrich (St. Louis, MI, USA), Ins], the above IR-A62 aptamer (Ori), and the two synthesized aptamers in combination (LD1+LD2) and alone (LD1 or LD2).

The phosphorylation of insulin receptors and signaling proteins was confirmed in insulin receptors-overexpressing Rat-1 fibroblasts (Rat-1/hIR).

The specific phosphorylation of tyrosine residues at Y960, Y1146, Y1150, Y1151, Y1316, and Y1322 of the insulin receptor (IR), the specific phosphorylation of the insulin receptor substrate (IRS, Y896), and phosphorylation of AKT and ERK, were confirmed.

The antibodies used in the Western blot were as follows:

Millipore: Anti-phospho-insulin receptor (Y1146), anti-phospho-insulin receptor (Y1150) and anti-phospho-tyrosine (4G10) antibody.

Invitrogen (Carlsbad, CA, USA): Anti-phospho-insulin receptor (Y1322), anti-phospho-insulin receptor (Y1316), anti-phospho-insulin receptor (Y1150/Y1151), and anti-phospho-insulin receptor (Y960) antibody.

Cell Signalling Technology (Danvers, MA, USA): Anti-phospho-ERK1/2 (T202/Y204), anti-phospho-AKT (T308) and anti-phospho-AKT (S473) antibody.

Invitrogen: Goat anti-rabbit IgG and anti-mouse IgG secondary antibodies conjugated to DyLight 800.

Western blots were performed to assess the phosphorylation of the proteins. Specifically, cells were seeded in 12-well plates, and for serum starvation, cells were incubated for 3 hours in FBS-free medium before stimulation with insulin or aptamer. The aptamer and insulin were then prepared in Krebs-Ringer HEPES buffer (25 mM HEPES (pH 7.4), 120 mM NaCl, 5 mM KCl, 1.2 mM MgSO₄, 1.3 mM CaCl₂, and 1.3 mM KH₂PO₄). To reconstruct the tertiary structure, the aptamer sample was heated at 95° C. for 5 minutes and then slowly cooled to room temperature. After stimulation with insulin (20 nM) or aptamer (100 nM), cells were washed three times with cold PBS and then lysed in lysis buffer. The cell lysate was then sonicated and centrifuged at 20,000×g for 15 minutes at 4° C., and the supernatant was mixed with 5× Laemmli sample buffer. After heating at 95° C. for 10 minutes, the proteins were separated on a Bis-Tris gel and transferred to a nitrocellulose membrane, and the membrane was incubated in blocking buffer (PBS, 5% nonfat dry milk, and 0.1% NaN₃) for 30 minutes at room temperature and then treated with the indicated antibodies overnight at 4° C. Finally, the membrane was washed three times in TTBS buffer (20 mM Tris, 150 mM NaCl, and 0.1% Tween 20) for 10 minutes each and incubated with secondary antibody for 1 hour at room temperature, followed by three more washes of the membrane with TTBS buffer for 10 minutes each. Then, the intensity of specific bands was analyzed using a LI-COR Odyssey infrared imaging system.

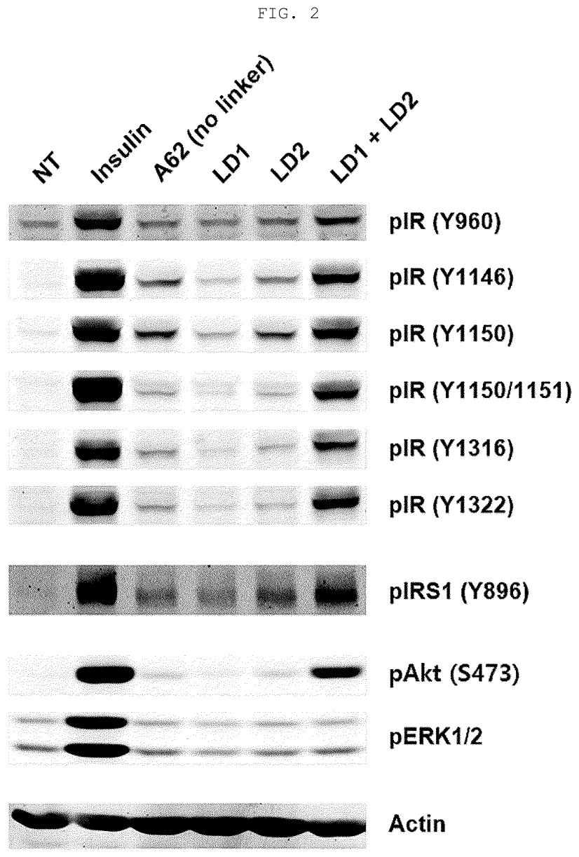

Results thereof are shown in FIG. 2.

As shown in FIG. 2, it was confirmed that treatment of the dimer (A62-LD1+LD2) significantly increased the level of phosphorylation on Y1150 when compared to the IR-A62 monomer (A62) or the aptamer containing the linker sequence (A62-LD1) or the sequence containing a complementary linker (A62-LD2). Further, it was found that the phosphorylation effect at positions Y960, Y1146, Y1150/10151, Y1316, and Y1322 of the insulin receptor and the phosphorylation effect at Y896 of the insulin receptor substrate occurred together. The increased phosphorylation at these other tyrosine residues showed characteristics equivalent to the phosphorylation expressed in insulin. Furthermore, when compared to the IR-A62 monomer, the IR-A62 dimer showed a very high effect on AKT phosphorylation, while the expression change on ERK was not significant.

### <Example 3> Optimization of Insulin Receptor-Binding Aptamer Dimer

For the optimization of the IR-A62 monomer (A62) capable of having a dimeric conformation, the length of the complementary sequence was optimized and it was confirmed for a single strand (monomer).

To optimize the lengths of the complementary sequences capable of forming dimers, dimers were constructed with linker sequences of 6 bp, 8 bp, 10 bp, 12 bp, 14 bp, 16 bp, 18 bp, and 20 bp in length. The linker sequences were randomly repeated A, C, T, and G sequences.

The linker sequence information for optimizing the lengths of the complementary sequences is shown in Table 3 below.

TABLE 3

| SEQ | Linker | Sequence (5′ → 3′) |
|---|---|---|
| ID NO | name | (with spacer) |
| 12 | (6 bp)-1 | TT GTGGCC |
| 13 | (6 bp)-2 | TT GGCCAC |
| 14 | (8 bp)-1 | TT ATTACAGC |
| 15 | (8 bp)-2 | TT GCTGTAAT |
| 16 | (10 bp)-1 | TT ATTACAGCTT |
| 17 | (10 bp)-2 | TT AAGCTGTAAT |
| 18 | (12 bp)-1 | TT ATTACAGCTTGC |
| 19 | (12 bp)-2 | TT GCAAGCTGTAAT |
| 20 | (14 bp)-1 | TT ATTACAGCTTGCTA |
| 21 | (14 bp)-2 | TT TAGCAAGCTGTAAT |
| 22 | (16 bp)-1 | TT ATTACAGCTTGCTACA |
| 23 | (16 bp)-2 | TT TGTAGCAAGCTGTAAT |
| 24 | (18 bp)-1 | TT ATTACAGCTTGCTACACG |
| 25 | (18 bp)-2 | TT CGTGTAGCAAGCTGTAAT |
| 26 | (20 bp)-1 | TT ATTACAGCTTGCTACACGAA |
| 27 | (20 bp)-2 | TT TTCGTGTAGCAAGCTGTAAT |

Then, similar to Example 2, to confirm the phosphorylation level of pY1150/1151 (10C3) to determine the length of a suitable complementary sequence, aptamers for the preparation of IR-A62 dimers were designed by linking the IR-A62 monomer (A62) of the modified SEQ ID NO: 7 above to the linkers in Table 3 above, and are shown in Table 4 below.

TABLE 4

| SEQ | | |
|---|---|---|
| ID | Name of | |
| NO | IR-A62 dimer | Sequence (5′ → 3′) |
| 28 | A62 D-1 (6 bp) | CANNACGCAN GAGNCNAGAN CCGN TT GTGGCC |
| 29 | A62 D-2 (6 bp) | CANNACGCAN GAGNCNAGAN CCGN TT GGCCAC |
| 30 | A62 D-1 (8 bp) | CANNACGCAN GAGNCNAGAN CCGN TT ATTACAGC |
| 31 | A62 D-2 (8 bp) | CANNACGCAN GAGNCNAGAN CCGN TT GCTGTAAT |
| 32 | A62 D-1 (10 bp) | CANNACGCAN GAGNCNAGAN CCGN TT ATTACAGCTT |
| 33 | A62 D-2 (10 bp) | CANNACGCAN GAGNCNAGAN CCGN TT AAGCTGTAAT |
| 34 | A62 D-1 (12 bp) | CANNACGCAN GAGNCNAGAN CCGN TT ATTACAGCTTGC |
| 35 | A62 D-2 (12 bp) | CANNACGCAN GAGNCNAGAN CCGN TT GCAAGCTGTAAT |
| 36 | A62 D-1 (14 bp) | CANNACGCAN GAGNCNAGAN CCGN TT ATTACAGCTTGCTA |
| 37 | A62 D-2 (14 bp) | CANNACGCAN GAGNCNAGAN CCGN TT TAGCAAGCTGTAAT |
| 38 | A62 D-1 (16 bp) | CANNACGCAN GAGNCNAGAN CCGN TT ATTACAGCTTGCTACA |
| 39 | A62 D-2 (16 bp) | CANNACGCAN GAGNCNAGAN CCGN TT TGTAGCAAGCTGTAAT |
| 40 | A62 D-1 (18 bp) | CANNACGCAN GAGNCNAGAN CCGN TT ATTACAGCTTGCTACACG |
| 41 | A62 D-2 (18 bp) | CANNACGCAN GAGNCNAGAN CCGN TT CGTGTAGCAAGCTGTAAT |
| 42 | A62 D-1 (20 bp) | CANNACGCAN GAGNCNAGAN CCGN TT ATTACAGCTTGCTACACGAA |
| 43 | A62 D-2 (20 bp) | CANNACGCAN GAGNCNAGAN CCGN TT TTCGTGTAGCAAGCTGTAAT |

Similar to Example 2 above, Western blots were performed to assess the phosphorylation of proteins.

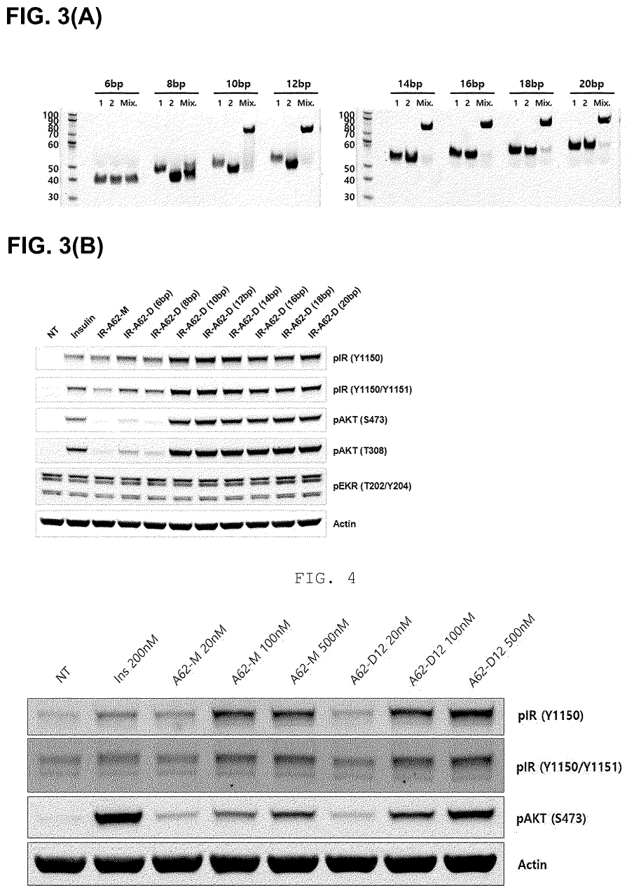

Results thereof are shown in FIGS. 3(A) and 3(B).

As shown in FIG. 3 (A), dimers were formed with linker lengths ranging from 6 to 20 bp.

Further, as shown in FIG. 3(B), with respect to the treatment of IR-A62 dimers (IR-A62-D(D1+D2) (6 bp to 20 bp)) when compared to IR-A62 monomers (IR-A62-M), IR-A62 aptamer dimers with linkers of 6 bp or 8 bp showed similar levels of Y1150 phosphorylation as IR-A62 aptamer monomers. On the other hand, IR-A62 aptamer dimers (10 bp to 20 bp) with linkers of 10 bp or more showed higher levels of Y1150 phosphorylation compared to IR-A62 aptamer monomers.

In addition, native gel analysis of IR-A62 aptamer dimers confirmed that IR-A62 aptamer dimers with a linking site length of 6 bp or 8 bp were not formed, but only IR-A62 aptamer dimers with a linking site length of 10 bp or more were formed.

These results suggest that IR-A62 aptamer dimers (6 bp or 8 bp) have a short linking site, which exist as monomers rather than forming dimers, and therefore do not have an elevated Y1150 phosphorylation level, whereas the IR-A62 aptamer dimers (10 bp to 20 bp) have an elevated effect on Y1150 phosphorylation level since they form dimers.

### <Example 4> Confirmation of Specific Phosphorylation of Tyrosine Residues on Insulin Receptors

Similar to Example 2, the hIR-L6 cell line was treated with IR-A62 aptamer monomer (IR-A62-M) or IR-A62 dimer (IR-A62-D (12 bp)) in a concentration-dependent manner, and then Western blots were performed to assess protein phosphorylation, similar to Example 2 above.

Results thereof are shown in FIG. 4.

As shown in FIG. 4, hIR-L6 cells were treated with IR-A62 aptamer monomer (A62-M) and IR-A62 aptamer dimer (12 bp) at different concentrations (20, 100, and 500 nM), and then the intracellular levels of pIR were measured by Western blotting. Next, IR-A62 aptamer dimer (12 bp) showed higher Y1150 phosphorylation levels and pAKT levels compared to IR-A62 aptamer monomer (the results of IR-A62 aptamer monomer 500 nM were similar to the results of IR-A62 aptamer dimer (12 bp) 100 nM).

### <Example 5> Confirmation of Changes in Glucose Uptake for Insulin Receptors

hIR-L6 Cells were treated with IR-A62 aptamer monomer (IR-A62-M) and IR-A62 aptamer dimer (12 bp) at different concentrations (20, 100, and 500 nM) and then treated with C14-2DG (C14 2-Deoxy-D-glucose), and glucose uptake was measured by liquid scintillation counter.

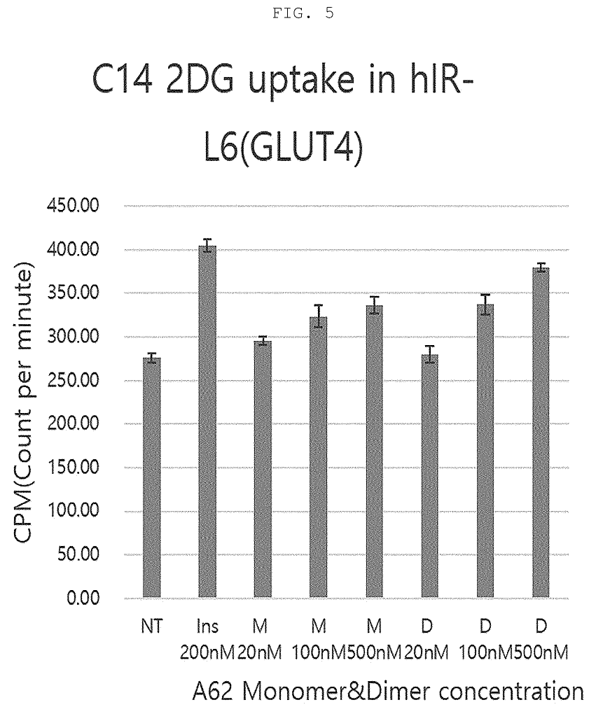

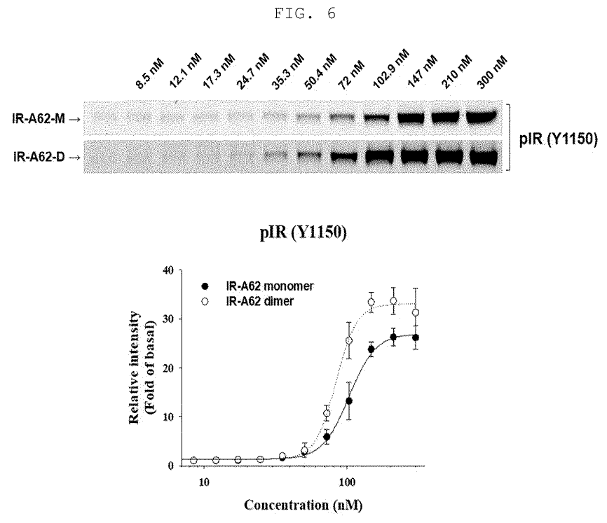

Results thereof are shown in FIG. 5.

As shown in FIG. 5, the glucose uptake effect by 100 nM of IR-A62 aptamer dimer (12 bp) was equivalent to that of 500 nM of IR-A62 aptamer monomer, and the glucose uptake effect by 500 nM of IR-A62 aptamer dimer (12 bp) was even higher, indicating that IR-A62 aptamer dimer (12 bp) had a higher glucose uptake effect than IR-A62 aptamer monomer.

### <Example 6> Confirmation of Changes in Tyrosine Residues (Y1150 and Total), AKT, and pERK Phosphorylation Levels for Insulin Receptors

Similar to Example 2, the Rat-1/IR cell line was treated with IR-A62 aptamer monomer (IR-A62-M) or IR-A62 dimer (IR-A62-D(20 bp)) in a concentration-dependent manner, and then Western blots were performed to assess phosphorylation levels and overall phosphorylation levels of Y1150 residue of the insulin receptor.

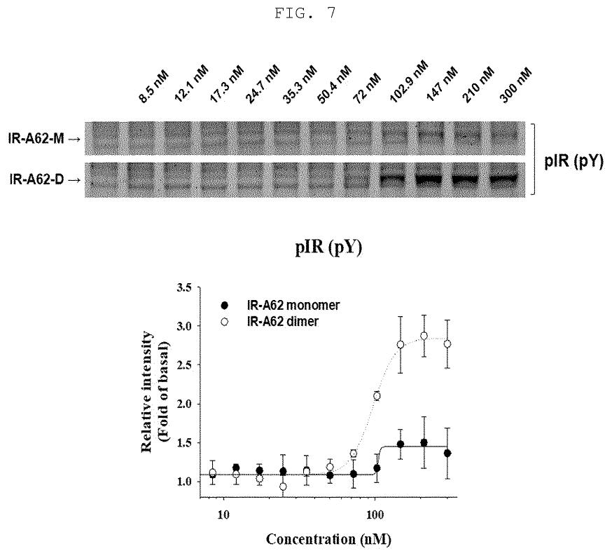

The results are shown in FIGS. 6 and 7.

As shown in FIG. 6, the IR-A62 aptamer dimer (IR-A62-D (20 bp)) according to the present disclosure exhibited significantly higher Y1150 residue-specific phosphorylation (IR-A62-M). Further, as compared to the monomeric aptamer shown in FIG. 7, in terms of overall phosphorylation, the IR-A62 aptamer dimer (IR-A62-D(20 bp)) according to the present disclosure exhibited superior phosphorylation efficacy compared to the monomer (IR-A62-M).

This confirms that the IR-A62 aptamer dimer (IR-A62-D (20 bp)) according to the present disclosure exhibits a better insulin receptor phosphorylation effect.

### <Example 7> Confirmation of Phosphorylation Level Changes for AKT and ERK

Similar to Example 2, the Rat-1/IR cell line was treated with IR-A62 aptamer monomer (IR-A62-M) or IR-A62 dimer (IR-A62-D (20 bp)) in a concentration-dependent manner, and then Western blots were performed to assess phosphorylation levels of AKT and ERK.

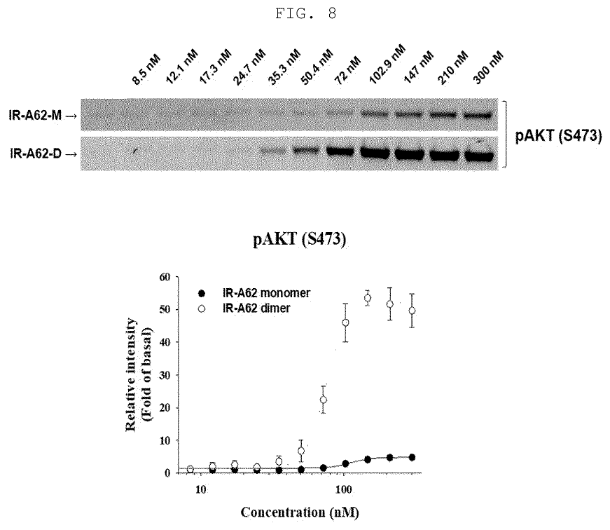

The results are shown in FIGS. 8 and 9.

As shown in FIG. 8, the level of pAKT was found to be significantly increased by the treatment with IR-A62 aptamer dimer.

On the other hand, as shown in FIG. 9, the phosphorylation level of ERK did not show a significant difference between the monomer and dimer.

In other words, upon considering its ability to enhance AKT levels without increasing the phosphorylation levels of ERK, IR-A62 aptamer dimer is acknowledged for its potential to address the risk of increased cancer incidence and atherosclerosis, both of which are concerning side effects of long-term insulin therapy.

### <Example 8> Confirmation of 41 Improved Effect by Introduction of 2′-OMe Modification to Linking Site Sequence of IR-A62 Aptamer Dimer

In the sequence of SEQ ID NO: 42 or 43 above, 2′-OMe (methoxy) substitution capable of providing nuclease resistance was performed on the linking site acting as a linker (linker sequences with SEQ ID NO: 26 and 27).

This is shown in SEQ ID NO: 44 and 45. These modification sequences added with 2′-OMe (methoxy) were labeled as 3′-inverted dT (3′-idT).

Rat-1/IR cell line was treated with insulin, IR-A62, and IR-A62 aptamer dimer of in SEQ ID NO: 45 and/or SEQ ID NO: 46 (2′-OMe), and the intracellular levels of pIR were measured by Western blotting.

TABLE 5

| SEQ | | | |
|---|---|---|---|
| ID | | | Linker |
| NO | Name | Sequence (5′→3′) | sequence |
| 44 | LD1 | CANNACGCAN GAGNCNAGAN | Bold, |
| | | CCGN TT ATTACAGCTTGCTACACGAA | underlined |
| | | idT | and |
| 45 | LD2 | CANNACGCAN GAGNCNAGAN | itralicized |
| | | CCGN TTTTCGTGTAGCAAGCTGTAAT | (2′-OMe |
| | | idT | substitution) |

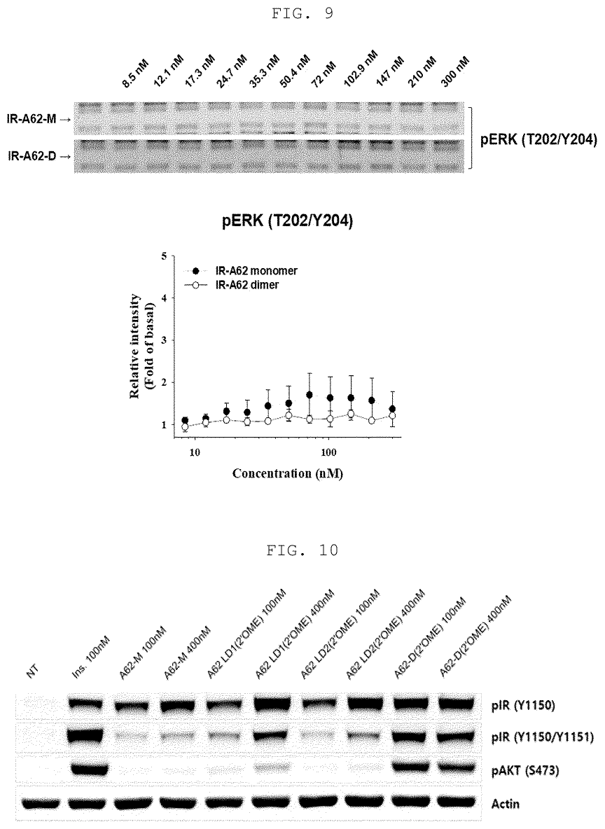

Results thereof are shown in FIG. 10.

As shown in FIG. 10, the IR-A62 aptamer dimer (A62-D (2′OMe); A62-LD1+LD2), in which the 2′-OMe modification was introduced into the linking site, had a superior effect on the phosphorylation of Y1150, Y1150/Y1151, and AKT of the insulin receptor, which exhibited a similar effect to insulin. In contrast, IR-A62 (A62-M), or the sequence with the linker sequence bound thereto (A62-LD1 or A62-LD2) did not achieve phosphorylation of Y1150, Y1150/Y1151, and AKT, or only acted at very low levels.

### <Example 9> Confirmation of Improved Half-Life of IR-A62 Aptamer Dimer

In-serum stability assay experiments were performed on IR-A62 monomer (no linker) and IR-A62 dimer (2′-OMe modification). Specifically, the aptamers in Table 5 above were incubated with 90% mouse serum at 37° C. for up to 48 hours each, and the degradation of the aptamers at different time points was analyzed using polyacrylamide gel electrophoresis.

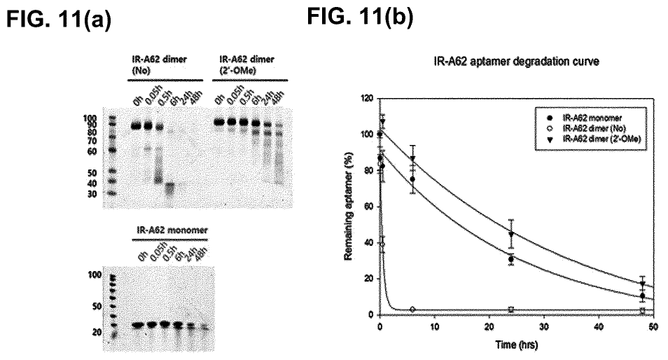

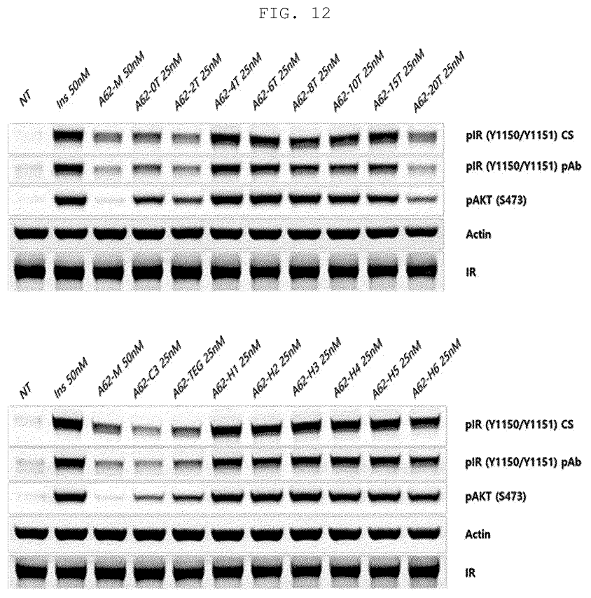

The results are shown in FIGS. 11(a) and 11(b).

As can be seen in FIG. 11(a), the aptamer in the dimeric form according to the present disclosure was found to remain stable in the blood for a period of time. When examined graphically, it was confirmed that the aptamer in the dimeric form exhibits significantly longer retention time in the blood compared to the monomeric form.

It was found from the above results that the stability of the aptamer dimer according to the present disclosure is significantly increased.

### <Example 10> Preparation of Novel IR-A62 Aptamer Dimer and Confirmation of its Effect

Meanwhile, unlike the conventional IR-A62 aptamer dimer synthesis method, an attempt was made to synthesize IR-A62 aptamer dimer with a single strand. Therefore, in order to make the distance between the two IR-A62 aptamer sequences shorter and more precisely controlled, efforts were made to identify the appropriate length for the spacer sequence.

The spacer sequence information is shown in Table 6 below.

TABLE 6

| SEQ | | |
|---|---|---|
| ID NO | Spacer Name | Sequence (5′ → 3′) |
| 46 | SSD (2T) | TT |
| 47 | SSD (4T) | TTTT |
| 48 | SSD (6T) | TTTTTT |
| 49 | SSD (8T) | TTTTTTTT |
| 50 | SSD (10T) | TTTTTTTTTT |
| 51 | SSD (15T) | TTTTTTTTTTTTTTT |
| 52 | SSD (20T) | TTTTTTTTTTTTTTTTTTTT |
| 53 | SSD (C3 | (Spacer Phosphoramidite |
| | phosphoramidite) | C3)₁ |
| 54 | SSD (TEG | (Spacer Phosphoramidite |
| | phosphoramidite) | C 9)₁ |
| 55 | SSD (HEG1 | (Spacer Phosphoramidite |
| | phosphoramidite) | C 18)₁ |
| 56 | SSD (HEG2 | (Spacer Phosphoramidite |
| | phosphoramidite) | C 18)₂ |
| 57 | SSD (HEG3 | (Spacer Phosphoramidite |
| | phosphoramidite) | C 18)₃ |
| 58 | SSD (HEG4 | (Spacer Phosphoramidite |
| | phosphoramidite) | C 18)₄ |
| 59 | SSD (HEG5 | (Spacer Phosphoramidite |
| | phosphoramidite) | C 18)₅ |
| 60 | SSD (HEG6 | (Spacer Phosphoramidite |
| | phosphoramidite) | C 18)₆ |

Then, similar to Example 2, to confirm the phosphorylation level of pY1150/1151 (10C3) to determine the length of a suitable complementary sequence, aptamers for 5 the preparation of IR-A62 dimers were designed by linking the IR-A62 monomer (A62) of the modified SEQ ID NO: 7 above to the linkers in Table 5 above, and are shown in Table 6 below. From the results of Example 10 above, the 2′-OMe modification demonstrated a superior effect on the phosphorylation of Y1150, Y1150/Y1151 and AKT of the insulin receptor that was comparable to insulin, and confirmed its stability in serum. Thus, in this design, the 2′-OMe modification of the spacer was introduced.

TABLE 7

| SEQ | Name of | |
|---|---|---|
| ID | IR-A62 | |
| NO | dimer | Sequence (5′→3′) |
| 61 | A62 SSD | CANNACGCAN GAGNCNAGAN CCGN TT TT CANNACGCAN |
| | (0T) | GAGNCNAGAN CCGN |
| 62 | A62 SSD | CANNACGCAN GAGNCNAGAN CCGN TT TT TT CANNACGCAN |
| | (2T) | GAGNCNAGAN CCGN |
| 63 | A62 SSD | CANNACGCAN GAGNCNAGAN CCGN TT TTTT TT CANNACGCAN |
| | (4T) | GAGNCNAGAN CCGN |
| 64 | A62 SSD | CANNACGCAN GAGNCNAGAN CCGN TT TTTTTT TT CANNACGCAN |
| | (6T) | GAGNCNAGAN CCGN |
| 65 | A62 SSD | CANNACGCAN GAGNCNAGAN CCGN TT TTTTTTTT TT CANNACGCAN |
| | (8T) | GAGNCNAGAN CCGN |
| 66 | A62 SSD | CANNACGCAN GAGNCNAGAN CCGN TT TTTTTTTTTT TT |
| | (10T) | CANNACGCAN GAGNCNAGAN CCGN |
| 67 | A62 SSD | CANNACGCAN GAGNCNAGAN CCGN TT TTTTTTTTTTTTTTT TT |
| | (15T) | CANNACGCAN GAGNCNAGAN CCGN |
| 68 | A62 SSD | CANNACGCAN GAGNCNAGAN CCGN TT TTTTTTTTTTTTTTTTTTTT TT |
| | (20T) | CANNACGCAN GAGNCNAGAN CCGN |
| 69 | A62 SSD | CANNACGCAN GAGNCNAGAN CCGN TT (Spacer Phosphoramidite C3)₁ TT |
| | (C3) | CANNACGCAN GAGNCNAGAN CCGN |
| 70 | A62 SSD | CANNACGCAN GAGNCNAGAN CCGN TT (Spacer Phosphoramidite 9)₁ TT |
| | (TEG) | CANNACGCAN GAGNCNAGAN CCGN |
| 71 | A62 SSD | CANNACGCAN GAGNCNAGAN CCGN TT (Spacer Phosphoramidite 18)₁ TT |
| | (HEG1) | CANNACGCAN GAGNCNAGAN CCGN |
| 72 | A62 SSD | CANNACGCAN GAGNCNAGAN CCGN TT (Spacer Phosphoramidite 18)₂ TT |
| | (HEG2) | CANNACGCAN GAGNCNAGAN CCGN |
| 73 | A62 SSD | CANNACGCAN GAGNCNAGAN CCGN TT (Spacer Phosphoramidite 18)₃ TT |
| | (HEG3) | CANNACGCAN GAGNCNAGAN CCGN |
| 74 | A62 SSD | CANNACGCAN GAGNCNAGAN CCGN TT (Spacer Phosphoramidite 18)₄ TT |
| | (HEG4) | CANNACGCAN GAGNCNAGAN CCGN |
| 75 | A62 SSD | CANNACGCAN GAGNCNAGAN CCGN TT (Spacer Phosphoramidite 18)₅ TT |
| | (HEG5) | CANNACGCAN GAGNCNAGAN CCGN |
| 76 | A62 SSD | CANNACGCAN GAGNCNAGAN CCGN TT (Spacer Phosphoramidite 18)₆ TT |
| | (HEG6) | CANNACGCAN GAGNCNAGAN CCGN |

### Bold, Underlined and Italicized: 2′-OMe Substitution

Similar to Example 2 above, Western blots were performed to assess the phosphorylation of proteins.

Results thereof are shown in FIG. 12.

As shown in FIG. 12, when compared to the A62 monomer (A62-M), treatment with the A62 dimers in Table 7 resulted in an increase in Y1150 phosphorylation levels for the IR-A62 aptamer dimers linked by Poly T, where the linking site sequence is between 4T and 15T, compared to the IR-A62 aptamer monomer.

On the other hand, the IR-A62 aptamer dimer connected by a short PEG linker showed an elevated Y1150 phosphorylation level, where the linking site sequence is between H1 and H6, compared to the IR-A62 aptamer monomer.

1. An aptamer dimer comprising:
(1) a first aptamer comprising: a sequence that specifically binds to an extracellular domain of an insulin receptor comprising the base sequence of SEQ ID NO: 1; and a linker sequence having a sequence complementary to a linker sequence of a second aptamer linked thereto, and
(2) the second aptamer comprising: a sequence that specifically binds to the extracellular domain of the insulin receptor comprising the base sequence of SEQ ID NO: 1; and a linker sequence having a sequence complementary to the linker sequence of the first aptamer linked thereto,
wherein SEQ ID NO: 1 is CANNACGCAN GAGNCNAGAN CCGN,
N is 5-(N-1-naphthylmethylcarboxamide)-2′-deoxyuridine or 5-(N-benzylcarboxamide)-2′-deoxyuridine,
C, A, or G is a modified or unmodified base,
the modified C is a modification of an introduction of a methoxy group (2-O-Me, 2-O-methoxy-DNA) at the C2 position of the nucleotide; or an introduction of fluorine (2-F, 2-fluorine-DNA) at the C2 position of the nucleotide,
the modified G is a modification of an introduction of a methoxy group (2-O-Me, 2-O-methoxy-DNA) at the C2 position of the nucleotide; or an introduction of fluorine (2-F, 2-fluorine-DNA) at the C2 position of the nucleotide, and
the modified A is a modification of an introduction of a methoxy group (2-O-Me, 2-O-methoxy-DNA) at the C2 position of the nucleotide; or an introduction of fluorine (2-F, 2-fluorine-DNA) at the C2 position of the nucleotide.

2. The aptamer dimer of claim 1, wherein the sequence that specifically binds to the extracellular domain of the insulin receptor comprising the base sequence of SEQ ID NO: 1 in the first aptamer and the second aptamer comprises 25 to 79 consecutive base sequences, comprising 22nd to 46th in the base sequence of SEQ ID NO: 2.

3. The aptamer dimer of claim 2, wherein the sequence that specifically binds to the extracellular domain of the insulin receptor comprising the base sequence of SEQ ID NO: 1 is any one selected from the group consisting of SEQ ID NO. 3 to 11.

4. The aptamer dimer of claim 1, wherein the linker sequence having the sequence complementary to the linker sequence of the second aptamer and the linker sequence having the sequence complementary to the linker sequence of the first aptamer form a dimer through complementary binding.

5. The aptamer dimer of claim 1, wherein the linker sequence has 5 to 30 base sequences.

6. The aptamer dimer of claim 5, wherein the linker sequence has at least one 2′-OMe (methoxy) or 2′-F (fluorine) modified base substitution on the base A, C, T, or G, or is L-form DNA or Locked DNA (LNA).

7. The aptamer dimer of claim 5, wherein the linker sequence is any one sequence selected from the group consisting of SEQ ID NO: 12 to 27.

8. The aptamer dimer of claim 1, wherein the first aptamer further comprises a spacer sequence between the linker sequence and the sequence that specifically binds to the extracellular domain of the insulin receptor comprising the base sequence of SEQ ID NO: 1.

9. The aptamer dimer of claim 8, wherein the spacer sequence is 1, 2, 3, 4, 5, or 6 poly T fragments.

10. The aptamer dimer of claim 1, wherein the first aptamer is any one selected from the group consisting of SEQ ID NO: 28, 30, 32, 34, 36, 38, 40, and 42.

11. The aptamer dimer of claim 1, wherein the second aptamer is any one selected from the group consisting of SEQ ID NO: 29, 31, 33, 35, 37, 39, 41, and 43.

12. The aptamer dimer of claim 1, wherein the aptamer dimer comprises any one of the first aptamer and the second aptamer selected from the group consisting of SEQ ID NO: 28 and 29, 30 and 31, 32 and 33, 34 and 35, 36 and 37, 38 and 39, 40 and 41, and 42 and 43.

13. An aptamer dimer comprising:
a first aptamer sequence that specifically binds to an extracellular domain of an insulin receptor comprising the base sequence of SEQ ID NO: 1;
a spacer sequence; and
a second aptamer sequence that specifically binds to the extracellular domain of the insulin receptor comprising the base sequence of SEQ ID NO: 1,
wherein the SEQ ID NO: 1 is CANNACGCAN GAGNCNAGAN CCGNC,
N is 5-(N-1-naphthylmethylcarboxamide)-2′-deoxyuridine or 5-(N-benzylcarboxamide)-2′-deoxyuridine,
C, A, or G is a modified or unmodified base,
the modified C is a modification with an introduction of a methoxy group (2-O-Me, 2-O-Methoxy-DNA) at the C2 position of the nucleotide; or an introduction of fluorine (2-F, 2-fluorine-DNA) at the C2 position of the nucleotide,
the modified G is a modification with an introduction of a methoxy group (2-O-Me, 2-O-Methoxy-DNA) at the C2 position of the nucleotide; or an introduction of fluorine (2-F, 2-fluorine-DNA) at the C2 position of the nucleotide, and
the modified A is a modification with an introduction of a methoxy group (2-O-Me, 2-O-Methoxy-DNA) at the C2 position of the nucleotide; or an introduction of fluorine (2-F, 2-fluorine-DNA) at the C2 position of the nucleotide.

14. The aptamer dimer of claim 13, wherein the sequence that specifically binds to the extracellular domain of the insulin receptor comprising the base sequence of SEQ ID NO: 1 is any one selected from the group consisting of SEQ ID NO: 3 to 11.

15. The aptamer dimer of claim 14, wherein the spacer sequence is a poly A fragment, a poly T fragment, a poly G fragment, a poly C fragment, a phosphoramidite fragment, or an ethylene glycol fragment.

16. The aptamer dimer of claim 15, wherein the spacer sequence is any one selected from the group consisting of SEQ ID NO: 46 to 60.

17. The aptamer dimer of claim 13, wherein the aptamer dimer is any one selected from the group consisting of SEQ ID NO: 61 to 76.

18. A method of preventing or treating a metabolic disease comprising administering the aptamer dimer of claim 1 to a subject in need thereof.

19. The method of claim 18, wherein the metabolic disease is any one selected from the group consisting of diabetes, idiopathic type 1 diabetes, latent autoimmune diabetes in adults, early-onset type 2 diabetes, young-onset atypical diabetes (YOAD), maturity-onset diabetes of the young (MODY), malnutrition-associated diabetes, gestational diabetes, hyperglycemia, insulin resistance, hepatic insulin resistance, impaired glucose tolerance, diabetic neuropathy, diabetic nephropathy, kidney diseases, diabetic retinopathy, adipocyte dysfunction, visceral fat accumulation, sleep apnea, obesity, weight gain due to the use of other medicaments, excessive sugar cravings, dyslipidemia, and non-alcoholic fatty liver disease (NAFLD).

20. The method of claim 18, wherein the metabolic disease is any one selected from the group consisting of diabetes, idiopathic type 1 diabetes, latent autoimmune diabetes in adults, early-onset type 2 diabetes, young-onset atypical diabetes (YOAD), maturity-onset diabetes of the young (MODY), malnutrition-associated diabetes, and gestational diabetes.
